(19) United States
(12) Patent Application Publication
(10) Pub. No.: US 2015/0322115 A1
(43) Pub. Date: Nov. 12, 2015

(54) MEANS AND METHODS FOR TREATING CMV

(71) Applicant: REDVAX GMBH, Schlieren (CH)

(72) Inventors: Sabine Wellnitz, Urdorf (CH); Corinne John, Horgen (CH); Christian Schaub, Waedenswil (CH)

(21) Appl. No.: 14/708,081

(22) Filed: May 8, 2015

(30) Foreign Application Priority Data
May 8, 2014 (LU) 92449
May 8, 2014 (LU) 92450
May 8, 2014 (LU) 92451
May 8, 2014 (LU) 92452

(51) Int. Cl. C07K 14/005; C12N 7/00; C12N 15/86; A61K 39/245

(52) U.S. Cl. CPC C07K 14/005; A61K 39/245; C12N 7/00; C12N 15/86; C12N 2710/16134; C12N 2710/16151

(57) ABSTRACT

The present invention relates to the field of recombinant protein production and vaccine preparation. In particular, the invention provides means and methods for producing the pentameric gH/gL/UL128/UL130/UL131A complex of CMV. More specifically, the invention provides a pentameric gH/gL/UL128/UL130/UL131A complex of CMV produced in a baculovirus system which can be used as a vaccine against CMV.

### BACKGROUND

Cytomegalovirus is a viral genus of the viral family known as Herpesviridae or herpesviruses. It is typically abbreviated as CMV. The species that infects humans is commonly known as human CMV (HCMV) or human herpesvirus-5 (HHV-5), and is the most studied of all cytomegaloviruses. About 60% of the adult population and in some countries 100% endemic infection is already reached. Infection is usually asymptomatic in immunocompetent subjects, while sometimes a mononucleosis-like illness may occur. The infection leads to the establishment of lifelong latency, which may occasionally be interrupted by reactivation. However, for immunodeficient subjects, such as cancer or transplant patients, neonates or HIV patients a CMV infection can be a serious threat, even resulting in mortality. CMV can persist in the host because the viral genome encodes multiple proteins that interfere with MHC class I presentation of viral antigens. One viral protein blocks translocation of peptides into the lumen of the endoplasmic reticulum, while two other viral proteins cause degradation of MHC class I proteins before they reach the cell surface.

Current treatments apply antiviral agents such as ganciclovir, foscarnet, acyclovir, cidofovir or leflunomide. While these agents can be suited for transplant patients and immunocompromised patients, they cannot be used for pregnant women. For these a hyperimmunoglobulin therapy is preferred. Though various attempts were made in the prior art to come up with a vaccine such a an attenuated live vaccine, a subunit protein vaccine, a subunit viral vector encoded vaccine, up to now, no vaccine is yet available.

Cytomegalovirus (CMV) is among the largest and most complex of the known viruses that cause human disease. The 235-kb genome encodes at least 165 proteins, but CMV vaccine research has focused on a limited number of viral proteins that dominate cellular or humoral immune responses during natural infection. The pp65 protein is a major target of the cytotoxic T-cell response. Located within the tegument between the capsid and the viral envelope, pp65 is the most abundant protein in CMV virions. The IE1 protein is also an important cytotoxic T-cell target that is not present in the virion but is abundantly expressed in cells after infection. On the virion surface and embedded in the envelope are several glycoprotein complexes that mediate host cell entry. A heterodimer comprised of glycoprotein M and glycoprotein N is believed to initiate host cell interaction by binding to heparin. A second heterodimer comprised of glycoprotein H and glycoprotein L (gH/gL) may mediate receptor interactions that culminate in the triggering of conformational changes in glycoprotein B (gB) that drive fusion of the viral envelope with the target cell membrane. A pentameric complex of CMV comprised of gH, gL, UL128, UL130, UL131A mediates entry into epithelial and endothelial cells. All of these complexes are important targets for humoral immunity as they contain epitopes that bind a select class of antibodies known as neutralizing antibodies. The overall goal in the development of a vaccine is to induce a neutralizing activity both against epithelial/endothelial cell infection and fibroblast infection by CMV.

A variety of vaccine approaches are under study, including simple peptides or subunits; recombinant multisubunit complexes such as gH/gL or the complete pentameric complex; inactivated CMV virions containing native pentameric complex, gB, pp65, and other viral antigens; genetically disabled CMV expressing native pentameric complex, which potentially combines the immunogenicity of a live vaccine with the safety of a killed vaccine; replication-defective viral vectors (eg, pox, adenovirus, alphavirus, and others) expressing subunits or multisubunit complexes; and prime/boost combinations of the above.

An attractive vaccine candidate is the pentameric complex of CMV that mediates entry into epithelial and endothelial cells. However, the extent to which conformational and/or multisubunit-dependent epitopes dominate the “neutralizing epitome” of the pentameric complex remains unclear. The possible necessity to represent the complete pentameric complex in its conformationally native state is suggested by a study of monoclonal antibodies isolated from naturally infected subjects: of 17 pentameric complex-specific neutralizing antibodies, all but 1 recognized multisubunit-dependent epitopes.

While many attempts to provide the pentameric complex of CMV, in particular HCMV were made in the prior art, to the best of the present inventors' knowledge, up to now the pentameric complex could not be provided in stable form and in sufficient amounts, while ideally having a high purity, as well. However, for the provision of a vaccine a stable pentameric complex in sufficient amounts with high purity that is immunogenic in order to induce an immune response is needed. The technical problem underlying the present invention is thus to satisfy this need.

The present invention solves this problem by providing novel means and methods for producing the pentameric complex of CMV. It is in particular based on the surprising finding that the pentameric complex can be stably expressed at high yield using a baculovirus vector in a suitable host cell, including insect cells. Moreover, the present inventors have unexpectedly found that the pentameric complex obtained by the methods of the invention is particularly useful as a pharmaceutical and/or immunogenic composition. This was clearly unforeseen, because recombinant proteins produced in insect cells differ from their “natural” counterparts with respect to their glycosylation pattern—an important parameter which modulates the immune response. Moreover, the present inventors have, at the first time, succeeded in providing a stable pentameric complex of CMV produced in an expression system, particularly in a baculovirus system, which allows for large-scale production of highly pure and immunogenic pentameric complexes.

### SUMMARY

The present inventors have pioneered in establishing new means and methods, which enable production of the pentameric complex of CMV in stable form and at high yield and purity. For the first time, the present inventors have expressed the protein components of the pentameric complex of CMV using a baculovirus vector in high quantities and observed assembly of a functional pentameric complex.

Thus, in a first aspect, the present invention relates to a pentameric complex composed of CMV proteins UL128, UL130, UL131A, gH (UL75) and gL (UL115) which is obtainable by the method, comprising
(i) co-expressing UL128, UL130, UL131A, gH (UL75) and gL (UL115) in a host cell by using baculovirus;
(ii) purifying the pentameric complex from host cells and/or supernatant obtained from said co-expression; and
(iii) optionally storing the purified pentameric complex in a buffer solution comprising a chelating agent and/or a stabilizing agent.

The host cell can be an insect cell or mammalian cell

The preferred production cell lines of this invention are insect cell lines such as Sf9, Sf21, Super Sf9-1 (VE-1), Super Sf9-2 (VE-2), Super Sf9-3 (VE-3), Hi-5, Mimic Sf9, Vankyrin, Express Sf+, and S2 Schneider cells, with Super Sf9-2 being preferred [Oxford Expression Technologies, Cat. No. 600103 and Fath-Goodin et al. (2006), Adv. Virus Res. 68, 75-90; Kroemer et al. (2006), J. Virol. 80(24), 12291-12228 and US20060134743. Super Sf-9 cells are engineered to stably express the Camoletis sonorensis ichnovirus P-vank-1 protein. For expression in mammalian cells, in particular human cells, e.g. HEK293, HEK293F, CHO, HeLa, HUVEC, HUAEC, Huh7, HepG2, BHK, MT-2, Cos-7, Cos-1, C127, 3T3, human foreskin fibroblasts (HFF), bone-marrow fibroblasts, Bowes melanoma, primary neural cells, or epithelial cells are used,

The co-expression step can include infecting host cells with a baculovirus expressing said proteins and having a titer of about 10⁷ pfu/mL or higher when infecting said host cell having a cell count at infection of about 2*10⁶ cells/mL; cultivating said host cells under suitable conditions, and harvesting said host cells and/or supernatant between 56-65 h post infection.

The host cells, in particular the insect cells, can be infected between day 15 and day 50, preferably between day 15 and day 30, preferably at day 18 after thawing and culturing.

Purification can include ion exchange chromatography, hydrophobic interaction chromatography, size exclusion chromatography and/or affinity chromatography.

The chelating agent can be Ethylenediaminetetraacetic acid (EDTA) or ethylene glycol tetraacetic acid (EGTA). Preferably, EDTA is present in said buffer solution at a concentration of 20 mM or less, such as 3 mM or less.

The stabilizing agent can be polyethylene glycol, arginine, glycine, sorbitol, trehalose, glycerol, sucrose, glucose, DMSO, TMAO and/or NP-40.

Further, the buffer solution can comprise Tris buffer, NaCl, MgCl₂, and/or KCl.

The open reading frames (ORFs) encoding CMV proteins UL128, UL130, UL131A, gH and gL can be on one or more vectors, preferably on a single vector.

The vector can contain elements for propagation in bacteria (E. coli), yeast (S. cerevisiae), insect cells and/or mammalian cells.

The ORFs can be located in the following order from 5′ to 3′ in said vector:

(i) gH, gL, UL128, UL130, UL131;

or (ii) gL, UL128, UL130, UL131, gH.

Preferably,
(a) in (i) the gH ORF is transcribed in 3′ direction, the gL ORF is transcribed in 5′ direction, the UL128 ORF is transcribed in 3′ direction, the UL130 ORF is transcribed in 3′ direction, and the UL131A ORF is transcribed in 3′ direction;
(b) in (i) gH ORF is transcribed in 3′ direction, the gL ORF is transcribed in 3′ direction, the UL128 ORF is transcribed in 3′ direction, the UL130 ORF is transcribed in 3′ direction, and the UL131A ORF is transcribed in 3′ direction;
(c) in (ii) gL ORF is transcribed in 5′ direction, the UL128 ORF is transcribed in 3′ direction, the UL130 ORF is transcribed in 3′ direction, the UL131A ORF is transcribed in 3′ direction, and the gH ORF is transcribed in 3′ direction.

Each of said ORFs can be driven by the p10 promoter, polh promoter, IE-1 promoter, mCMV promoter, vp39 promoter, lef2 promoter, CAG promoter, HepB SV40 promoter or any other promoter described herein and followed by a terminator sequence such as HSVtk terminator or SV40 terminator or any other terminator described herein.

At least one of said proteins can comprise a tag, which can be a His-Tag, Strep-Tag, a His-Strep-tag, StrepII-Tag, Softag 1, TC-tag, myc-Tag, FLAG-tag, HA-tag, V5-tag, Avi-tag, Calmodulin-tag, polyglutamate-tag, amyloid beta-tag, GST-tag, MBP-tag or S-tag. Preferably, the gH protein is equipped with a His-Tag, preferably comprising 8 His-residues.

One or more of said proteins can comprise PRESCISSION protease (GE Healthcare Life Sciences) or PRESCISSION and TEV protease, preferably the gH and/or gL protein comprises PRESCISSION protease or PRESCISSION and TEV protease. PRESCISSION Protease is a genetically engineered fusion protein consisting of human rhinovirus 3C protease and GST. It specifically cleaves between the Gln and Gly residues of the recognition sequence of LeuGluValLeuPheGln/GlyPro. The TEV protease is a highly site-specific cysteine protease that is found in the Tobacco Etch Virus (TEV).

The baculovirus genes v-cath and/or ChiA activity can be functionally disrupted.

The pentameric complex of the invention can also be in the form of a composition.

The pentameric complex of the invention can also be capable of inducing neutralization activity that inhibits both epithelial/endothelial (Epi/EC) and fibroblast infection.

In a second aspect, the present invention also provides a method for the production of a pentameric complex composed of CMV proteins UL128, UL130, UL131A, gH (UL75) and gL (UL115), comprising (i) co-expressing baculovirus CMV proteins UL128, UL130, UL131A, gH (UL75) and gL (UL115) in a host cell; (ii) purifying the pentameric complex from host cells and/or supernatant obtained from said co-expression; and (iii) optionally storing the purified pentameric complex in a buffer solution comprising a chelating agent and/or a stabilizing agent.

In a third aspect, a pharmaceutical composition or immunogenic composition comprising a pentameric complex of the invention or obtainable by the method of the invention and optionally a pharmaceutically acceptable carrier or adjuvant is provided.

In a fourth aspect, in the pentameric complex of the invention, at least one, two, three or four of said proteins can be derived from a CMV strain other than the CMV strain from which the remaining proteins are derived from.

The CMV proteins can be derived from CMV strain Towne, Towne having the genome as deposited with NCBI GenBank under accession number FJ616285.1, Toledo (GU937742.1), AD169 (FJ527563), Merlin (AY446894.2), TB20/E (KF297339.1), VR1814 (GU179289). The aa Y at position 204 was exchanged by the aa F, according to Patrone et al., J. Virol., 2005, 79, 8361-8373 the major problem of expression of UL130 is the frameshift at the same position leading to an amino acid expansion to the next ORF. In a fifth aspect, the invention provides a modified CMV Towne strain having the genome as deposited with NCBI GenBank under accession number FJ616285.1 and having at position 204 of the amino acid sequence of the UL130 ORF the amino acids F as well as the repair of frameshift at the same position of the labstrain Towne (grown on human foreskin fibroblasts) resulting in a functional amino acid Y.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

The term “less than” or “greater than” includes the concrete number. For example, less than 20 means less than or equal to. Similarly, more than or greater than means more than or equal to, or greater than or equal to, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### DESCRIPTION OF THE FIGURES

FIG. 1: Schematic representations of recombinant vectors for expression of CMV-pentameric complex and soluble CMV proteins.

The different variants are inserted into the vector backbone pRBT136 aimed at recombinant protein expression using the baculovirus expression system (BEVS) and containing two promoters P1 and P2 ( [symbol] _p10, [symbol] _polh) and two terminator sequences T1 and T2 (T), which are SV40 and HSVtk. For propagation in yeast the vectors contain an origin of replication (O), e.g. 2 micron, and a marker gene (m), e.g. URA3. Furthermore the vectors contain the transposon sites left (TL) and right (TR) for transposition of the transgenes from the transfer vector into bacmids, a loxP site (L) for site specific homologous recombination (plasmid fusion), origins of replication (O), ampicillin (A), chloramphenicol (C) and gentamycin (G) resistance genes, and defined restriction sites. For the expression in mammalian cells, either by transduction with a baculovirus or transient expression, the vector backbone pRBT 393 contains in addition a promoter selected from pCMV, ie1 and lef2, and a terminator selected from SV40 pA, BHG pA and HSVtk.

Abbreviations: c: consensus sequence; H: His-tag; SH: Streptavidin-His-tag; V: strain VR1814, pcl: precission protease, pcll: precission and TEV protease, DT: dimerization tool, T: terminator, O: origin of replication, G: gentamycin resistance, C: chloramphenicol resistance, L: loxP site, TL: left transposon side, TR: right transposon side.

FIG. 2: Analysis of purification process consisting of affinity chromatography steps, followed by size exclusion chromatography of the His-tagged soluble CMV-pentameric complex.

The pentameric complex comprising the surface proteins UL75 (His-tagged, gH)-UL115 (gL)-UL128-UL130-UL131A (SEQ ID NO: 18) was purified by an affinity based chromatography (IMAC) using His-Trap columns, followed by size exclusion chromatography (XK16/60 Superdex200 pg).

(A) 2^{nd} IMAC purification was analysed by SDS-PAGE (4-12% Bis-Tris gel) followed by coomassie-staining. The sizes (kDa) of the protein standard (lane 1) are marked on the left side. Lane 2: pool of 1^{st} IMAC (load of the 2^{nd} IMAC process), lane 3: flowthrough, lane 4: wash, lanes 5-14 representing elution fractions with increasing imidazole concentration up to 500 mM.

(B) Analysis of size exclusion chromatography purification by SDS-PAGE (4-12% Bis-Tris gel) followed by coomassie-staining. The sizes (kDa) of the protein standard (lane 1) are marked on the left side. Lane 1: elution pool IV-2, precipitated; lane 2: elution pool IV-2, non precipitated; lane 3: elution pool IV-3; lane 4: elution pool V; lane 5: elution pool VI; lane 6: size marker. Indicated by an arrow are the proteins of the pentameric complex (gH, gL, UL128, UL130, UL131A).

(C) Immunoblot using antibody against the His-tag of gH. Lane 1 represents the elution pool IV-2 (precipitated); lane 2: elution pool IV-2 (non precipitated); lane 3: elution pool IV-3; lane 4: elution pool V; lane 5: elution pool VI; lane 6: positive control. The His-tagged gH protein is marked on the right side.

(D) Coomassie stained SDS-PAGE (4-12% Bis-Tris gel) of purified, soluble human CMV (HCMV) pentameric complex (SEQ ID NO: 18) (lane 6). For determination of protein concentration by densitometric analysis different amounts of BSA were loaded (lane 2: 0.1 mg/mIL lane 3: 0.2 mg/mL; lane 4: 0.3 mg/mL; lane 5: 0.5 mg/mL). The sizes (kDa) of the protein standard (lane 1) are marked on the left side.

(E) The pentameric complex comprising the surface proteins UL75 (His-tagged, gH)-UL115 (gL)-UL128-UL130-UL131A (SEQ ID NO: 18) was purified by a one step affinity based chromatography (IMAC) using a Ni2+ charged column (scale dependent on bulk size), followed by concentration on a PALL macrosep centrifugal device and dialysis against storage buffer containing (25 mM Tris, 150 mM NaCl, 3 mM KCL, pH 6.5, 0.2% Brij-35) The purified, soluble complex was analyzed by SDS-PAGE (4-12% Bis-Tris gel) followed by Coomassie staining and densitomertric analysis alongside of different amounts of BSA (lane 2: 0.1 mg/mIL lane 3: 0.2 mg/mL; lane 4: 0.3 mg/mL; lane 5: 0.5 mg/mL; lane 6: purified pentameric complex). The sizes (kDa) of the protein standard (lane 1) are marked on the left side.

FIG. 3: Neutralization assay to verify humoral immune response based on SEQ ID NO:18

Comparison of neutralizing antibody titers of human CMV blood donors and mouse sera. Serum from five positive (Group G2, D6-D10) and five negative (Group G1, D1-D5) HCMV blood donors in a two-fold serial dilution (1:20 to 1:2560) were subjected to a cell-based fluorescence neutralization assay. The serum dilution (s.d.) that gave the same inhibitory effect as a 1:320 dilution of pooled sera obtained from eight mice previously immunized with the pentameric complex (SEQ ID NO:18) is shown. The separate analysis of the soluble pentameric complex, as one component in the antigen mix used for immunization of mice, showed the induction of neutralizing antibodies in comparison to human blood donors. PR: pentamer pre-immune, PO: pentamer post-immune, G1: negative blood donors, G2: positive blood donors.

FIG. 4: Quality control of the soluble complex, SEQ ID NO: 18, as vaccine candidate.

Fractions eluted with different amounts of imidazole are shown after being subjected to an IMAC-based purification. They were tested for the presence of UL75 (gH) and the His tag on gH. The similarity in signal intensity designates the intactness of gH and therefore of the whole complex composed of UL75-UL115-UL128-UL30-UL131A.

1: load, 2: flowthrough, 3: wash, 4: 250 mM imidazole, 5-8: 300 mM imidazole, 9-13: 350 mM imidazole, 14: positive control, 15: negative control

FIG. 5: Quality control of the soluble complex, SEQ ID NO: 18, as vaccine candidate using conformation-dependent antibodies.

Different production batches of the complex were tested with a sandwich ELISA assay for the co-presence of the designated proteins. Samples were captured with an anti-gH1 (UL75) antibody and detected with an anti-gH2 and an anti-His as well as anti-UL130/131A and anti-UL130 conformation-dependent (UL130/UL131A) antibodies. The signals confirm co-existence of the proteins in a complex, intactness of the complex, as well as reproducibility of its production.

1: batch 451-pool 1, 2: batch 459-pool 2, 3: batch 459-pool 1 (15 mM EDTA), 4: batch 459-pool 1 (20 mM EDTA), 5: batch 458 (15 mM EDTA), 6: positive control, 7: negative control, 8: internal standard

FIG. 6: Reference amino acid sequences for CMV gH, gL, UL128, UL130 and UL131A proteins.

gH (SEQ ID NO: 1), gL (SEQ ID NO: 2), UL128 (SEQ ID NO: 3), UL130 (SEQ ID NO: 4), UL131A (SEQ ID NO: 5)

FIG. 7: Qualitative overview of cellular immune response based on SEQ ID NO:18 and SEQ ID NO:67

(A) Comparison of cytokine secretion showing Th-1 and Th-2 response dependent on the antigen which is used for immunization of mice. (B, C, D) The spleenocytes were re-stimulated with a AD169 virus lysate whereas the cytokine secretion was verified by a multiplex assay according to manufacturer's protocol. The adjuvant led to an increase of IL-4 secretion whereas it had no stimulatory effect of the secretio of IFN-gamma or IL-5. The cytokine secretion is dose dependent for the pentameric complex; lower dosage was beneficial.

P1: pentameric complex (Towne strain); P2: pentameric complex (Towne and VR1814 strain); adj: adjuvant; VLP: virus like particle; gB: soluble glycoprotein gB (UL55); BV: baculovirus

FIG. 8: Neutralization assay to verify humoral immune response based on SEQ ID NO:18 and SEQ ID NO:67

Comparison of neutralizing antibody titers of human CMV blood donors and mouse sera. Serum from five positive (seropositive) and five negative (seronegative) HCMV blood donors in a two-fold serial dilution (1:20 to 1:2560) were subjected to a cell-based fluorescence neutralization assay. The serum dilution (s.d.) that gave the same inhibitory effect as a 1:100 dilution of pooled sera obtained from four out of eight mice previously immunized with the pentameric complex (SEQ ID NO:18, SEQ ID NO:67) in combination with VLP, gB and adjuvant is shown. The separate analysis of the soluble pentameric complex, as one component in the antigen mix used for immunization of mice, showed the induction of neutralizing antibodies in comparison to seropositive and seronegative human blood donors. The neutralization of fibroblast (MRC-5) and epithelial cells (ARPE-19) were investigated with two different virus strain, the VR1814 and TB40E strain.

P1: pentameric complex (Towne strain); P2: pentameric complex (Towne and VR1814 strain); adj: adjuvant; VLP: virus like particle; gB: soluble glycoprotein gB (UL55); BV: baculovirus; preimmune: before immunization; immune: post immunization

FIG. 9: Analysis of purification process consisting of affinity chromatography steps, and ion-exchange chromatography of the His-tagged soluble CMV-pentameric complex

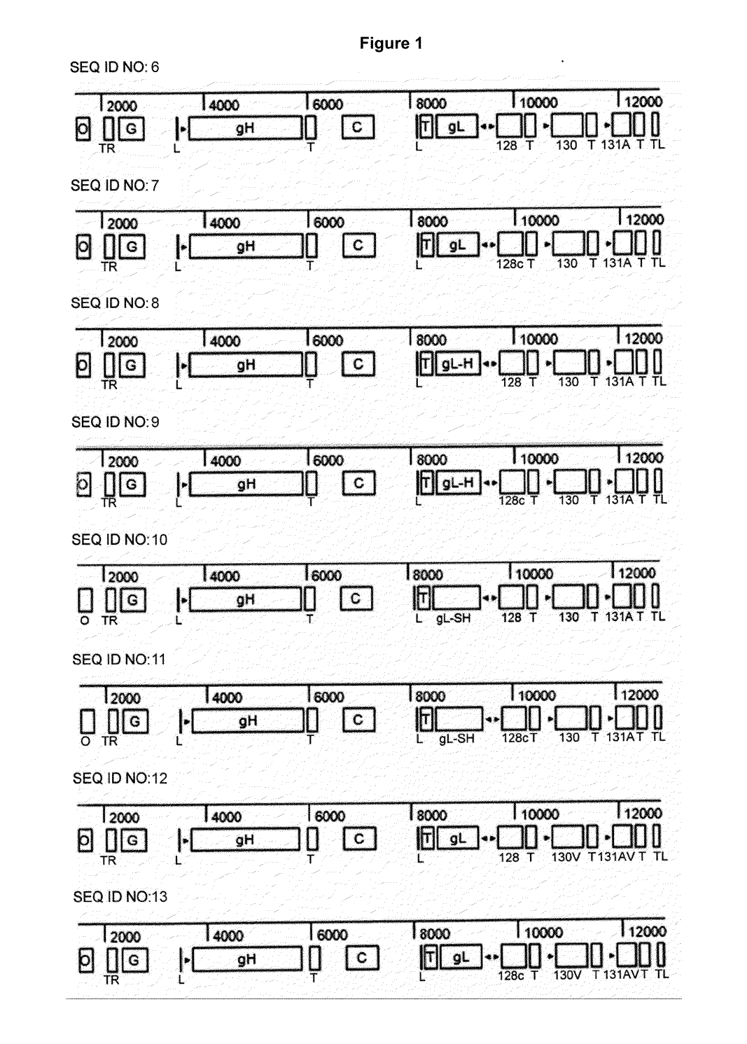

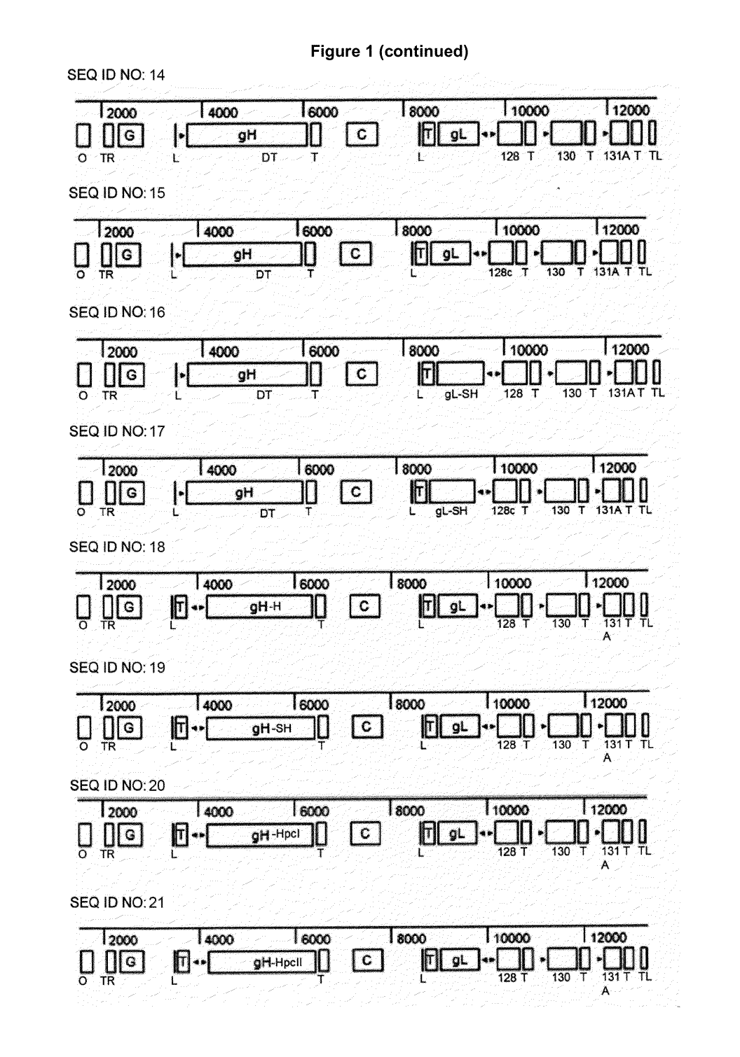

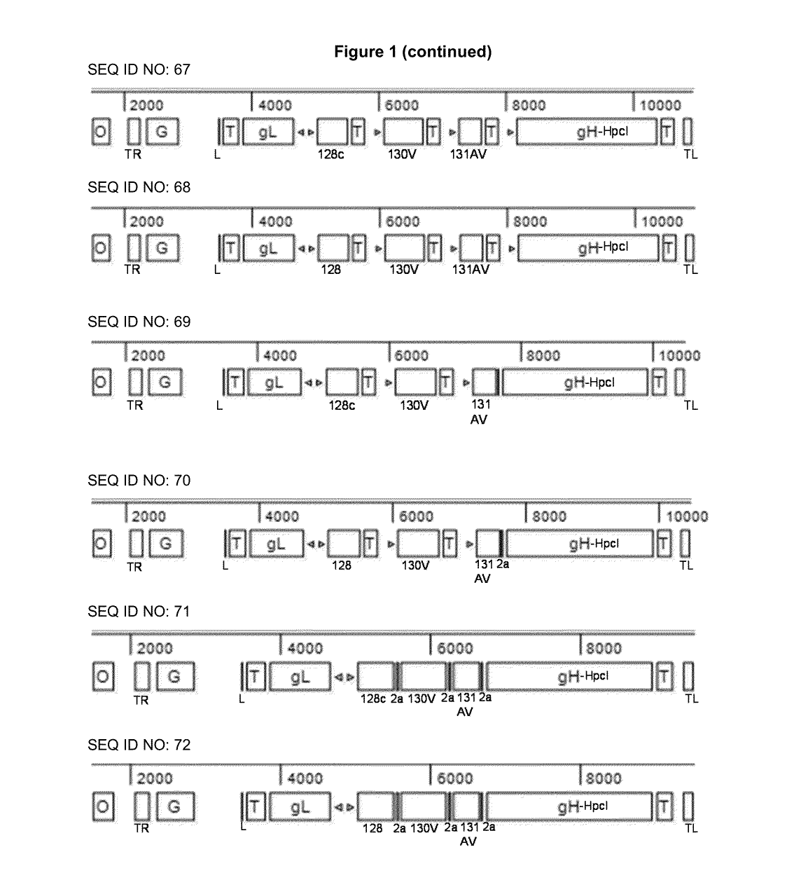

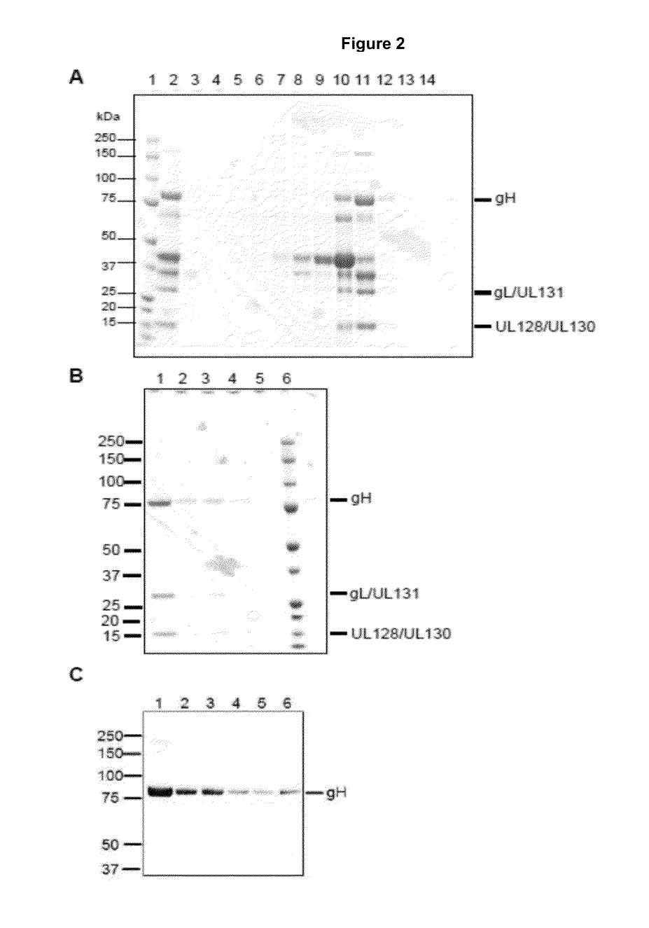

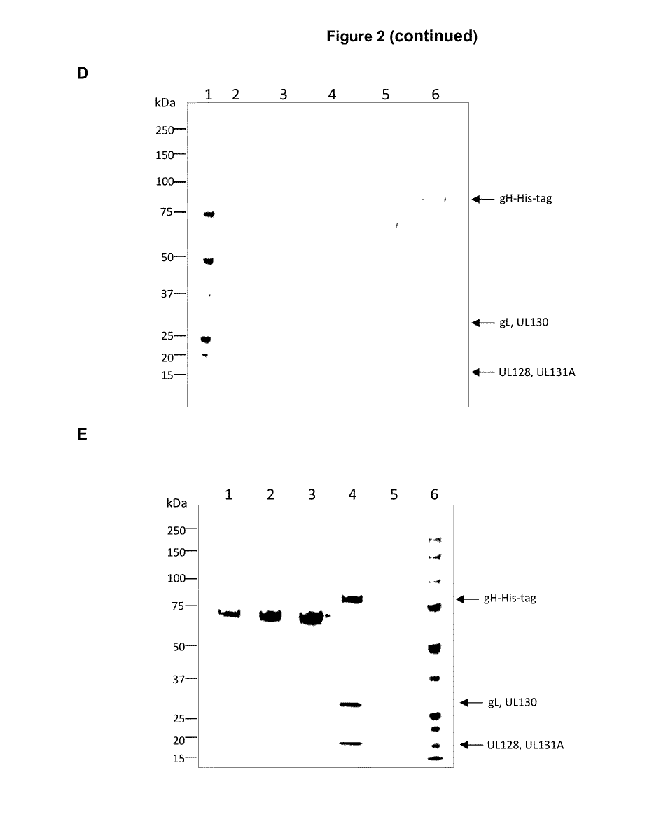

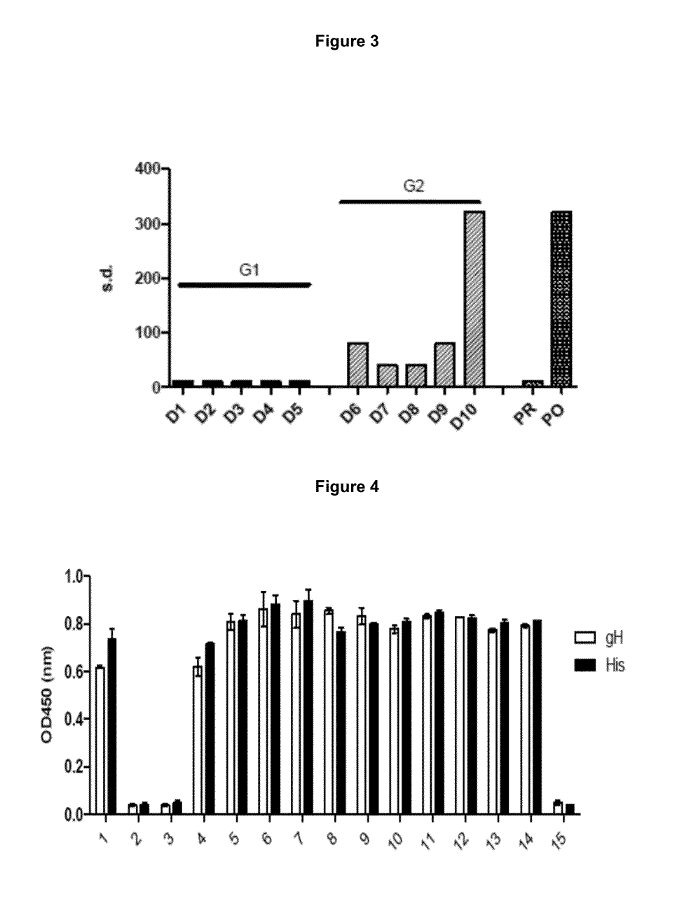

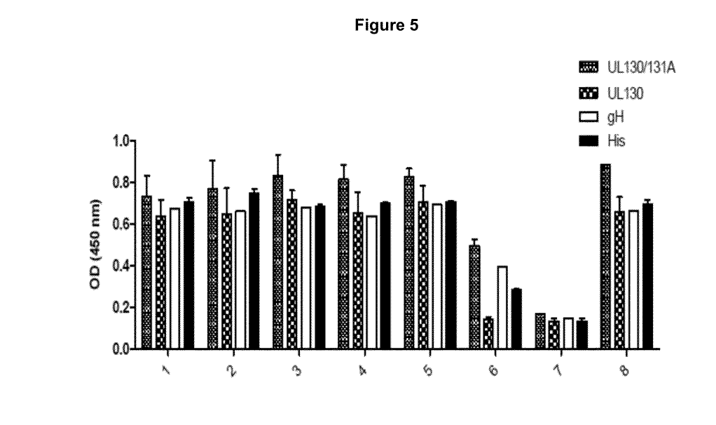

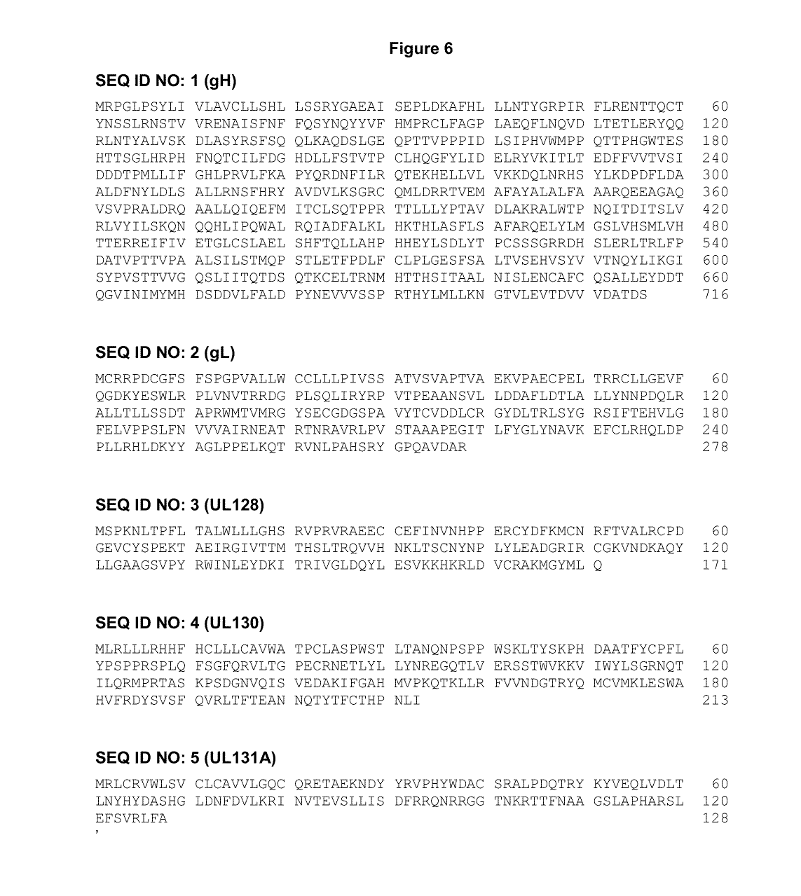

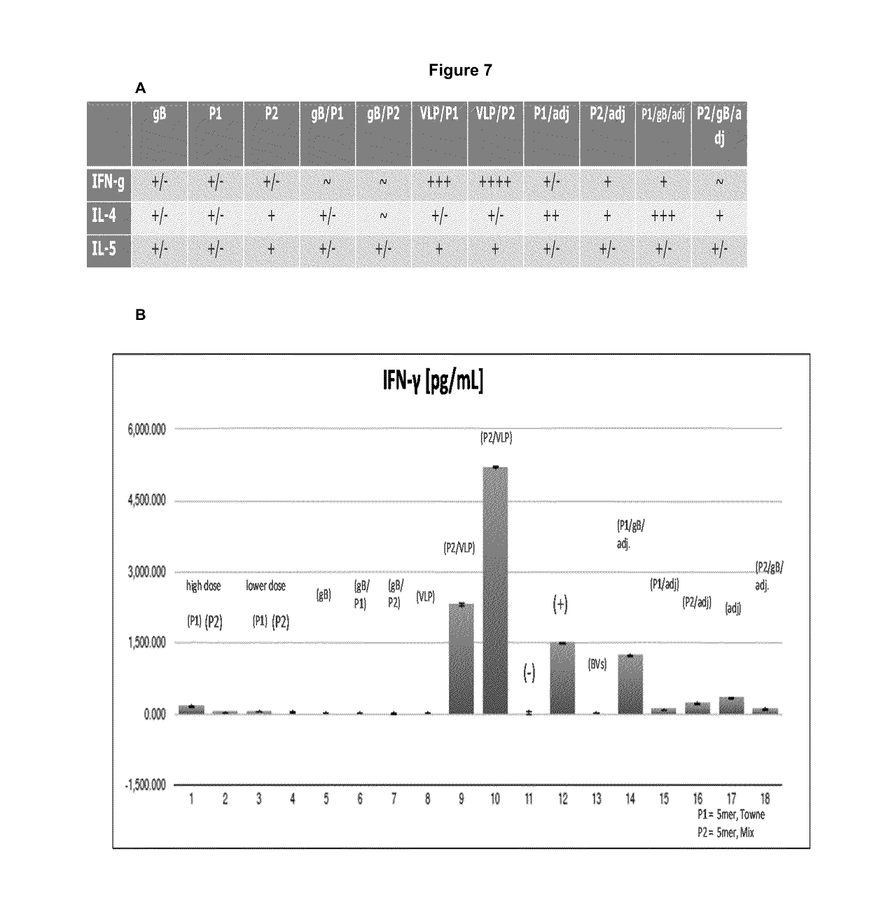

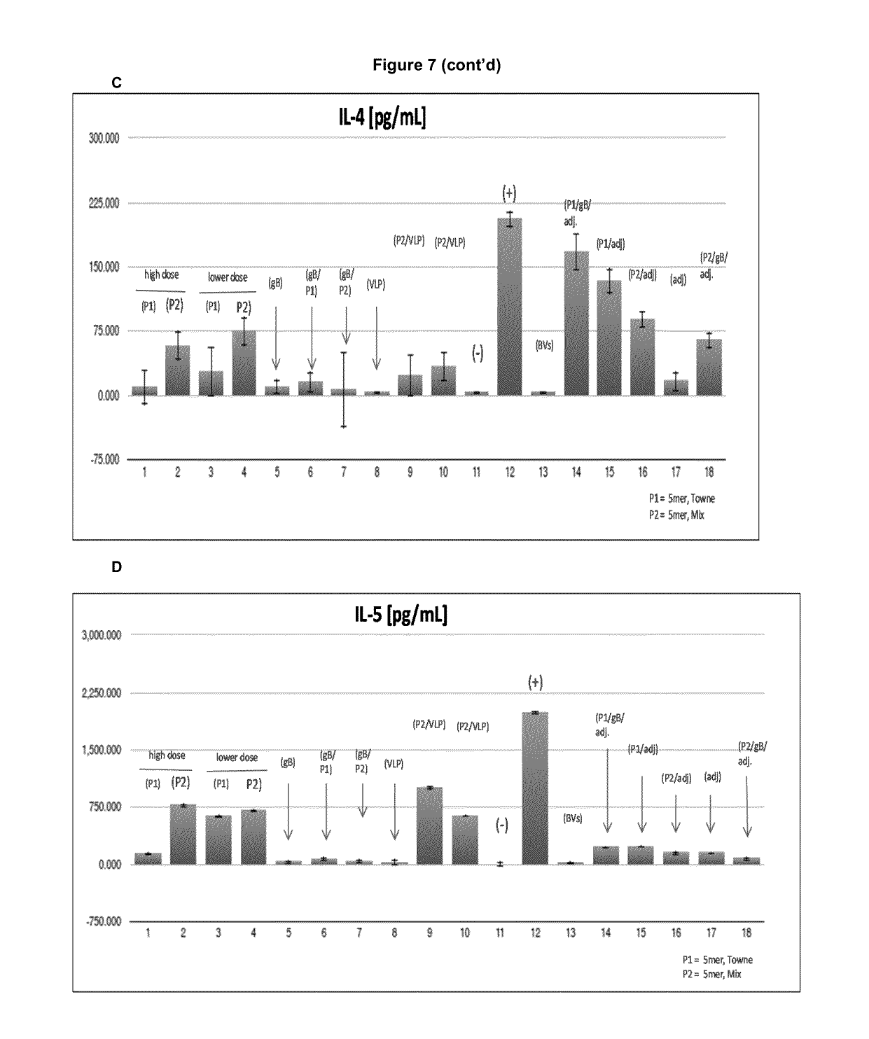

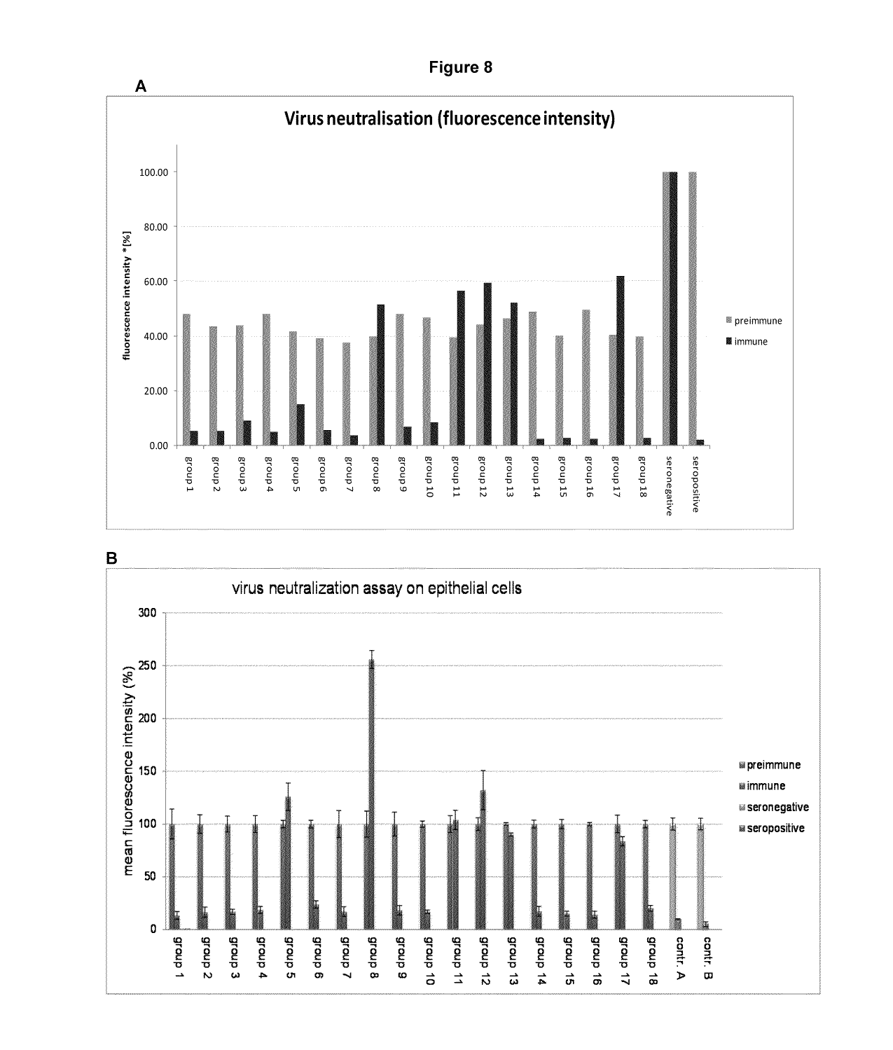

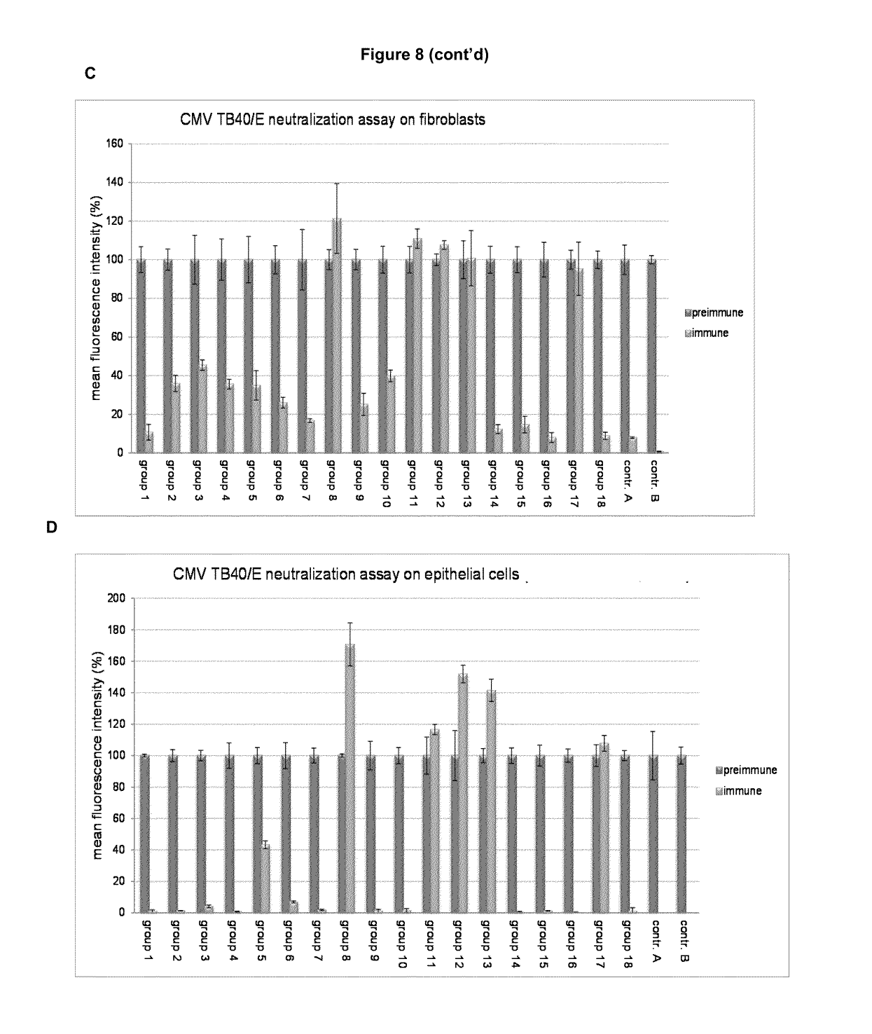

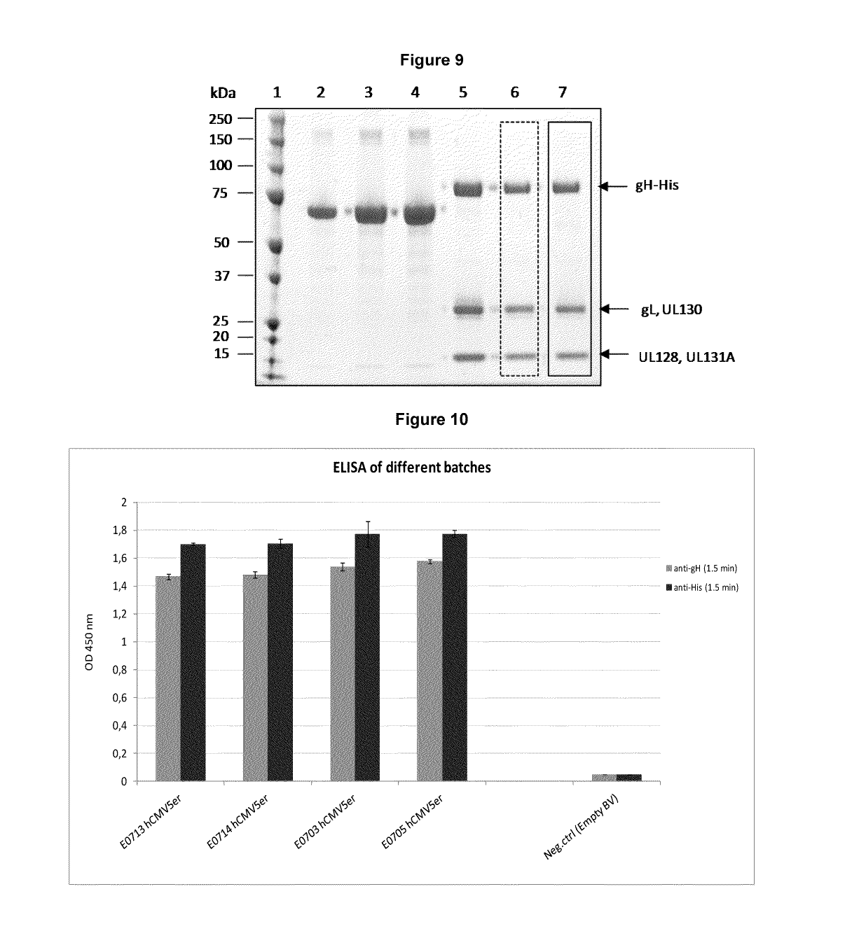

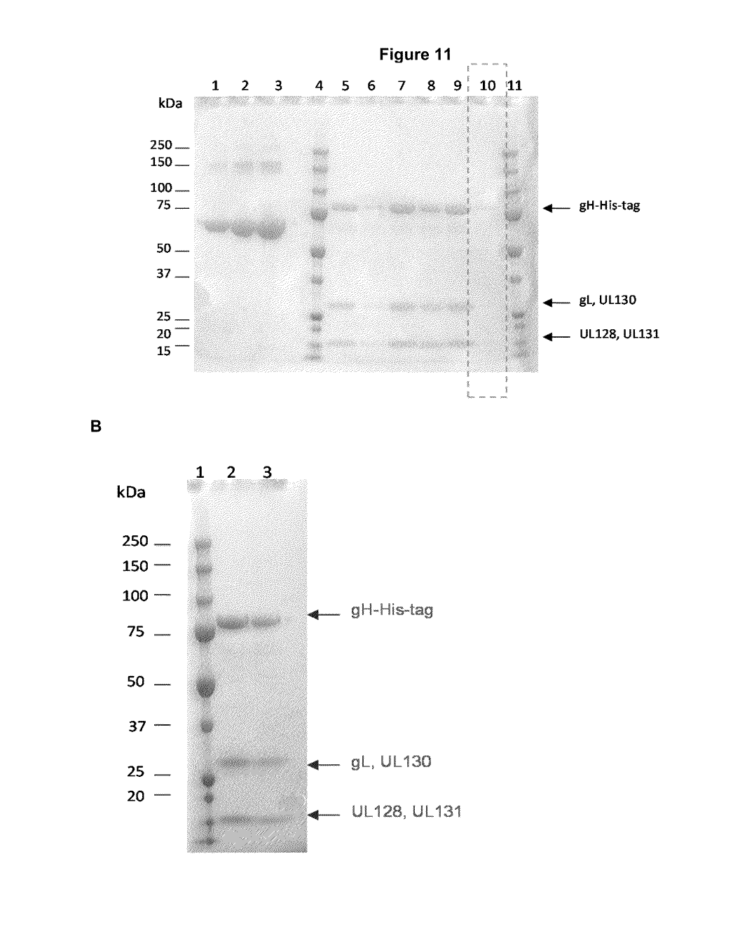

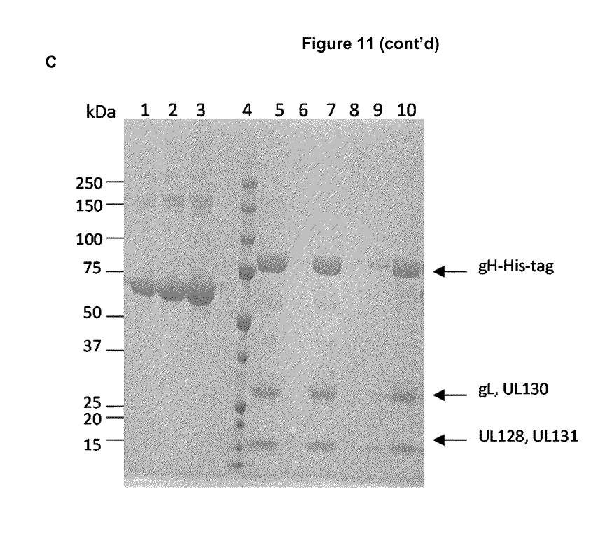

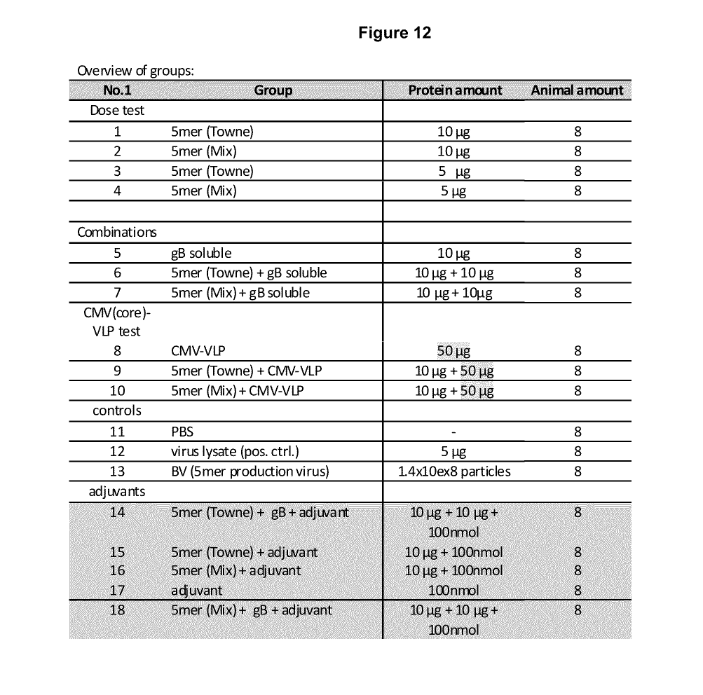

Coomassie blue (SimplyBlue, Invitrogen) stained SDS-PAGE (NuPAGE Invitrogen) of soluble human CMV (HCMV) pentameric complex (batch E0713, lane 6 and batch E0714, lane 7) was used for densitometric analysis (ImageJ software) of purity and concentration. The three bands observed in FIG. 1 represent gH-His as well as the co-migrating gL/UL130 and UL128/UL131A proteins (all identified in previous batches by mass spectrometry). The sum of the three bands was compared to a BSA standard (densitometric analysis; ImageJ software). For determination of protein concentration by densitometric analysis different amounts of BSA were loaded (lane 2: 0.2 mg/mL; lane 3: 0.4 mg/mL; lane 4: 0.6 mg/mL). The HCMV pentameric complex concentration on final samples (lanes 6 and 7) was measured, respectively for E0713 and E0714. *MM: Precision Plus Protein™ all blue standards (Bio-Rad, #161-0373, lane 1).

FIG. 10: Characterization of purified pentameric complex based on SEQ ID NO:18.

Product identity, at the end of the DSP process (IMAC-AEX-IMAC), was confirmed via a direct ELISA. The complex was verified with an α-gH-antibody (Santa Cruz, sc-58113) and an α-His-antibody (AbD Serotec, MCA1396) and detected with an α-mouse-HRP antibody (Cell Signaling, 7076S). The signals confirm co-existence of gH and the tag and therefore point to the intactness of the soluble HCMV pentameric complex. The negative control (neg.ctrl.) reflects a baculovirus sample devoid of the genes-of-interest.

FIG. 11: Influence of stabilizing agents in regard to yield of pentameric complex based on SEQ ID NO:18.

Verification of pentameric complex stability in regard to buffer, pH and stabilizing reagents such as EDTA. After purification dialysis with various buffers based on previous thermofluor-shift assays were performed to verify the influence of stabilizing agents in combination with different buffers and pH. For determination of protein concentration by densitometric analysis different amounts of BSA were loaded (lane 1: 0.2 mg/mL; lane 2: 0.4 mg/mL; lane 3: 0.6 mg/mL). The HCMV pentameric complex in the supernatant and pellet after dialysis with different buffers were shown. Lane5: PBS, 20 mM EDTA, pH 6.0 (supernatant); lane 6: PBS, 20 mM EDTA, pH 6.0 (pellet); lane 7: Tris, 20 mM EDTA, pH 7.4 (supernatant); lane 8: Tris, 20 mM EDTA, pH 7.4 (pellet); lane 9: Tris, 20 mM EDTA, pH 6.0 (supernatant); lane 10: Tris, 20 mM EDTA, pH 6.0 (pellet); *MM: Precision Plus Protein™ all blue standards (Bio-Rad, #161-0373, lane 4).

(B, C) Validation of various amounts EDTA during dialysis. The usage of 10 mM EDTA showed clearly that ca. half of the product was lost by precipitation (pellet), increasing amount of EDTA stabilized the complex. (B) pentameric complex with low amount of EDTA, lane 2: Tris, 10 mM EDTA, pH 7.4 (supernatant); lane 3: Tris, 10 mM EDTA, pH 7.4 (pellet); (C) For determination of protein concentration by densitometric analysis different amounts of BSA were loaded (lane 1: 0.2 mg/mL; lane 2: 0.4 mg/mL; lane 3: 0.6 mg/mL). The HCMV pentameric complex in the supernatant and pellet after dialysis with different buffers were shown. Lane5: Tris, 20 mM EDTA, pH 7.4 (supernatant); lane 6: Tris, 20 mM EDTA, pH 7.4 (pellet); lane 7: Tris, 15 mM EDTA, pH 7.4 (supernatant); lane 8: Tris, 15 mM EDTA, pH 7.4 (pellet); lane 9: Tris, 25 mM EDTA, pH 6.0 (pellet); lane 10: Tris, 25 mM EDTA, pH 6.0 (supernatant); *MM: Precision Plus Protein™ all blue standards (Bio-Rad, #161-0373, lane 4). The buffer with minimal precipitation effect for the 1st dialysis step seems to be Tris, 20 mM EDTA, pH 7.4.

FIG. 12: Schedule for in vivo study

A further in vivo study was performed to verify dose effects, pentameric complex variants and combinations with various CMV proteins either with or without an adjuvant

### DETAILED DESCRIPTION

There is an ongoing need to identify potent CMV antigens that elicit protective, neutralizing immune responses to CMV, and to develop CMV vaccines that achieve high protection levels. The pentameric gH/gL/UL128/UL130/UL131A complex of CMV is a promising tool for developing novel CMV vaccines. However, large-scale production of the pentameric complex has been hampered by inefficient protein expression systems. The present inventors have, for the first time, used a baculovirus system to co-express the protein components, and reported that a functional pentameric complex was assembled which could elicit an immunogenic response in vivo.

Thus, in a first aspect, the present invention provides a pentameric complex composed of CMV proteins UL128, UL130, UL131A, gH (UL75) and gL (UL115) which is obtainable by the method, comprising
(i) co-expressing CMV proteins UL128, UL130, UL131a, gH (UL75) and gL (UL115) in a host cell by using baculovirus;
(ii) purifying the pentameric complex from host cells and/or supernatant obtained from said co-expression; and
(iii) optionally storing the purified pentameric complex in a buffer solution comprising a chelating agent and/or a stabilizing agent.

The term “CMV” refers to Cytomegalovirus, a viral genus of the Herpesviridae or herpesviruses. In general, the term encompasses all species of CMV, including, inter alia, Human cytomegalovirus (HCMV), which is also known as Human herpesvirus 5 (HHV-5), Chimpanzee cytomegalovirus (CCMV), Simian cytomegalovirus (SCCMV) and Rhesus cytomegalovirus (RhCMV). Preferably, the CMV in accordance with the present invention is HCMV. A large number of strains of HCMV are known, including but not limited to TR, Towne, AD 169, Toledo, Merlin, TB40, Davis, etc.

Typically, CMV comprises at least 5 capsid proteins (gene products of UL46, UL48A, UL85, UL86, UL104), 19 regulatory proteins, 17 tegument proteins (gene products of UL25, UL45, UL47, UL48, UL69, UL71, UL72, UL76, UL77, UL83 [pp65], UL88, UL93, UL94, UL95, UL97, UL99, UL103), 5 surface or envelope proteins (gene products of UL55 [gB], UL73 [gN], U74 [gO], UL75 [gH], UL100 [gM], UL115 [gL]), the non-categorized gene products from the open reading frame UL128, UL130, UL131A; proteins from 15 beta-herpesvirus specific genes (UL23, UL24, UL32, UL33, UL35, UL36, UL38, UL43, UL74 [gO], UL78, UL82, UL96, IRS1, US22, TRS1) and so-called functional proteins from the open reading frames (ORF) UL50, UL80.5.

The term “pentameric complex” or in its short form as also used herein “complex” refers to a protein complex comprising the five CMV proteins UL128, UL130, UL131A, gH (UL75) and gL (UL115) which is thought to facilitate virus entry into target cells, in particular endothelial, epithelial and fibroblast cells. A model of the pentameric complex and its protein-protein interactions has been proposed by Ryckmann B J et al. J Virol. 2008; 82(1): 60-70. In the pentameric complex of the invention, gH, gL and pUL128 are thought to be typically linked through disulfide bonds, and UL130 and UL131A are typically incorporated into the pentameric complex (and/or inter-linked) by non-covalent interactions. The stochiometrics of the pentameric complexes are assumed to be 1:1:1:1:1 (Ryckmann B J et al. J Virol. 2008; 82(1): 60-70). It is preferred that the pentameric complex of the invention is able to elicit antibodies in vivo which immunologically cross-react with a CMV virion. The pentameric complex is preferably soluble. However, it may also be in membrane-bound form, although this is less preferred. Solubility is preferably achieved by deleting the transmembrane domain of gH. The term “composed of” when used in the context of the pentameric complex of the present invention means that the pentameric complex encompasses/comprises the five proteins gH, gL, UL128, UL130 and UL131A as described herein and, may, in addition, encompass further CMV proteins. However, preferably, the pentameric complex contains only the five CMV proteins gH, gL, UL128, UL130 and UL131A. The pentameric complex of the present invention may be in the form of a composition. Accordingly, one or more additional agents are added to or admixed with the pentameric complex, thereby resulting in a composition. Such additional agents are described herein, e.g. a buffer, chelating agent and/or a stabilizing agent. Suitable compositions are further described herein.

In the pentameric complex of the invention, one or more of the proteins can comprise additional B- and/or T-cell epitopes. Said T-cell epitope can be a CD4 T-cell epitope or a CD8 T-cell epitope. Preferably, said epitope is any one of the epitopes shown in SEQ ID NOs: 22-66.

An “epitope” is the part of an antigen that is recognized by the immune system, e.g. B cells or T cells. The term encompasses both conformational and linear (or sequential) epitopes. Conformational epitopes comprise discontinuous sections of the antigen's amino acid sequence, whereas linear epitopes are composed of a continuous section of the antigen's amino acid sequence. The term further includes cryptotopes and neotopes. “Cryptotopes” are epitopes which are hidden in the naturally occurring antigen, e.g. virus, but can become accessible when the antigen is not present in its natural conformation. “Neotopes” are epitope found only in quaternary structures of proteins, but not in protein monomers.

It is envisaged that additional epitopes may be fused to the amino acid sequence of one or more protein components of the pentameric complex. Fusion of amino acid sequences to the desired protein component(s) of the complex of the invention can be achieved by standard methods of genetic engineering well known to the person skilled in the art.

In accordance with the present invention, the epitope can be a B-cell and/or a T-cell epitope.

B cell epitopes is a region of an antigen (e.g., a native protein) recognized by either a particular membrane-bound B-cell receptor (BCR) or an antibody. A number of methods are readily available to identify or select B-cell epitopes, including x-ray crystallography, array-based oligopeptide scanning, site-directed mutagenesis, mutagenesis mapping, and phage display, as well as computational methods as reviewed by Sun et al. Comput Math Methods Med. 2013; 2013: 943636. For example, suitable methods include as structure-based prediction models, which rely on the 3D structure of antigen and epitope-related propensity scales, including geometric attributes and specific physicochemical properties. Structure-based algorithms and web servers (programs) include, e.g., EPSVR & EPMeta (http://sysbio.unl.edu/services/), EPCES (http://sysbio.unl.edu/services/EPCES/), and Epitopia (http://epitopia.tau.ac.il/). Mimotope-based prediction methods are combinatorial methods which require both antibody affinity-selected peptides and the 3D structure of antigen as input. Exemplary algorithms and programs based on mimotope-based prediction models include, e.g., MimoPro (http://informatics.nenu.edu.cn/MimoPro), PepSurf (http://pepitope.tau.ac.il and EpiSearch (http://curie.utmb.edu/episearch.html). Further, sequence-based prediction models are available which only rely on the primary sequence of an antigen, e.g. BEST and Zhang's method as reviewed in Sun et al. Comput Math Methods Med. 2013; 2013: 943636. In addition, binding sites prediction models can be used which infer methods that that focus on binding sites prediction of protein-protein interaction the interaction of an antigen and an antibody, e.g. ProMate, ConSurf, PINUP, and PIER.

Without wishing to be bound by a specific theory, it is envisaged that the presence of one or more B-cell epitopes in the complex of the invention preferably has an immunostimulatory effect on B-cells e.g., which results in activation and/or differentiation of the B cell and elicits an immunogenic response, which may e.g. result in the generation of neutralizing antibodies. B cells can be tested with different methods according to standard protocols known in the art to determine the immunostimulatory potential of an epitope. Suitable assays lymphoproliferation assays, detection of activation markers induced on specific T cells, ELIspot, intracytoplasmatic cytokine staining (ICS), and Cytokine Secretion.

T-cell epitopes are typically derived from processed protein antigens. In the context of the present invention, the T cell epitope can be a CD4-T cell epitope or a CD8 T-cell epitope. While cytotoxic (CD8) T-cells recognize intracellular peptides displayed by MHC class I molecules (CD8 T-cell epitopes), T helper cells recognize peptides that are taken up from the extracellular space and displayed by MHC class II molecules (CD4 T-cell epitopes). The peptide:MHC complex (pMHC) interacts with the T-cell receptor, leading to its activation and subsequent induction of a cellular immune response.

A number of in silico methods for T cell epitope prediction and/or selection are available. For CD8+ T cell epitope prediction, NetCTL-1.2 (http://www.cbs.dtu.dk/services/NetCTL/), EpiJen (http://www.ddg-pharmfac.net/epijen/EpiJen/EpiJen.html), or MAPPP (http://www.mpiib-berlin.mpg.de/MAPPP/), can be used, as reviewed in Larsen et al. BMC Bioinformatics 2007, 8:424. For CD4+T cells, computational models for epitope prediction have been reviewed by Oyarzún P et al. BMC Bioinformatics 2013, 14:52 and include data-driven methods which rely on peptide sequence comparisons to identify binding motifs, e.g. Rankpep (http://imed.med.ucm.es/Tools/rankpep.html), TEPITOPE, and NN-align (http://www.cbs.dtu.dk/services/NNAlign/), as well as structure-based methods which perform molecular modeling calculations in order to estimate the binding energies, thus offering independence from experimental binding data, e.g. NetMHCIIPan-2.0 (http://www.cbs.dtu.dk/services/NetMHCIIpan-2.0/), TEPITOPEpan (http://www.biokdd.fudan.edu.cn/Service/TEPITOPEpan/), and Predivac (http://predivac.biosci.uq.edu.au/).

Without wishing to be bound by a specific theory, it is envisaged that the presence of one or more T-cell epitopes in the complex of the invention preferably has an immunostimulatory effect on T-cells, e.g., which results in activation and/or differentiation of the T cell and preferably elicits an immunogenic response. Suitable methods to determine the immunostimulatory potential of an epitope on T cells include MHC peptide multimer assays, Solid Phase MHC-Peptide Complex assays, lymphoproliferation assays, detection of activation markers induced on specific T cells, ELIspot, intracytoplasmatic cytokine staining (ICS), Cytokine Secretion and Cell Surface Capture (CSC), and Cytokine Secretion and Well Surface Capture (Cell-ELISA), as reviewed in Li Pira G et al. J Biomed Biotechnol. 2010; 2010:325720.

In the alternative to the pentameric complex as described herein, it is also envisaged that other CMV complexes are produced by the means and methods of the present invention and are thus applied in the aspects and embodiments of the present invention. For example, it is envisaged that a complex composed of two, three or four of gH, gL, UL128, UL130 and UL131A is produced. A preferred example of a dimeric complex is a complex between gH and gL or UL130 and UL131A. Another preferred example of an alternative to the pentameric complex is a trimeric complex between gH/gL/gO. Hence, all aspects and embodiments described herein in connection with the pentameric complex are fully applicable to the dimeric or trimeric complexes described before, mutatis mutandis. It is also envisaged that other CMV complexes described herein can comprise additional B- and/or T-cell epitopes. Said T-cell epitope can be a CD4 T-cell epitope or a CD8 T-cell epitope as described herein.

Complexes of the invention are preferably prepared and used in isolated form. The term “isolated” as used herein means removed from its natural environment. Hence, an “isolated pentameric complex” or “isolated complex” does preferably not encompass the CMV membrane protein complex on the surface of CMV infected cells or within an infectious CMV virion.

The term “gH” when used herein may sometimes be referred to as “UL75” or “pUL75”. Each of these terms can replace the other and, thus, these terms are used interchangeably. The term “gH” encompasses gH polypeptides having mutations relative to the reference sequence shown in SEQ ID NO: 1 and also encompasses polypeptides having an amino acid sequence which shares a certain degree of identity with the amino acid sequence shown in SEQ ID NO: 1 as described herein. Also encompassed by said term are fragments of gH polypeptides having a length of 50, 100, 150, 200, 250, 300, 350, 400, 450, 500, 550, 600, 650, or 700 amino acids, whereby said fragments are preferably capable of forming a pentameric complex with the other four proteins as described herein. A preferred gH polypeptide lacks the transmembrane domain (TM). The absence of a TM domain means that this modified polypeptide cannot reside within a lipid bilayer. In some embodiments, the gH polypeptide lacks the full-length natural TM domain; in other embodiments, it can retain a portion of the natural TM domain, but not enough to let the protein reside in a lipid bilayer. Thus the polypeptide can contain up to 10 amino acids (e.g. 1, 2, 3, 4, 5, 6, 7, 8, 9 or 10 amino acids) of the natural gH TM domain. In addition to lacking some or all of the TM domain, the polypeptide may also lack the natural C-terminal domain of CMV gH or may lack a portion of the C-terminal domain.

The ectodomain of gH corresponds to the portion of gH which lacks the hydrophobic transmembrane domain (TM). The location and length of the ectodomain, the signal sequence and the TM domain can be predicted based on computational analysis of the hydrophobicity along the length of a given gH protein sequence. The signal sequence and the TM domain have the highest levels of hydrophobicity and these two regions flank the ectodomain, which is less hydrophobic.

The term “gL” when used herein may sometimes be referred to as “UL115” or “pUL115”. Each of these terms can replace the other and, thus, these terms are used interchangeably. The term “gL” encompasses gL polypeptides having mutations relative to the reference sequence shown in SEQ ID NO: 2 and also encompasses polypeptides having an amino acid sequence which shares a certain degree of identity with the amino acid sequence shown in SEQ ID NO: 2 as described herein. Also encompassed by said term are fragments of gL polypeptides having a length of 50, 100, 150, 200, or 250 amino acids. whereby said fragments are preferably capable of forming a pentameric complex with the other four proteins as described herein.

The term “UL128” when used herein may sometimes be referred to as “pUL128”. Each of these terms can replace the other and, thus, these terms are used interchangeably. The term “UL128” encompasses UL128 polypeptides having mutations relative to the reference sequence shown in SEQ ID NO: 3 and also encompasses polypeptides having an amino acid sequence which shares a certain degree of identity with the amino acid sequence shown in SEQ ID NO: 3 as described herein. Also encompassed by said term are fragments of UL128 polypeptides having a length of 50, 100, or 150 amino acids whereby said fragments are preferably capable of forming a pentameric complex with the other four proteins as described herein.

The term “UL130” when used herein may sometimes be referred to “pUL130”, or “UL130A”. Each of these terms can replace the other and, thus, these terms are used interchangeably. The term “UL130” encompasses UL130 polypeptides having mutations relative to the reference sequence shown in SEQ ID NO: 4 and also encompasses polypeptides having an amino acid sequence which shares a certain degree of identity with the amino acid sequence shown in SEQ ID NO: 4 as described herein. Also encompassed by said term are fragments of UL130 polypeptides having a length of 50, 100, 150, or 200 amino acids whereby said fragments are preferably capable of forming a pentameric complex with the other four proteins as described herein.

The term “UL131A” when used herein may sometimes be referred to “pUL131A”, “UL131” or “pUL131”. Each of these terms can replace the other and, thus, these terms are used interchangeably. The term “UL131a” encompasses UL131A polypeptides having mutations relative to the reference sequence shown in SEQ ID NO: 5 and also encompasses polypeptides having an amino acid sequence which shares a certain degree of identity with the amino acid sequence shown in SEQ ID NO: 5 as described herein. Also encompassed by said term are fragments of UL131A polypeptides having a length of 50, or 100 amino acids whereby said fragments are preferably capable of forming a pentameric complex with the other four proteins as described herein.

As stated each protein of the invention, in particular, gH, gL, UL128, UL130 and UL131A, respectively, or a fragment thereof, may contain mutations, such as insertions, deletions and substitutions relative to the reference sequences shown in SEQ ID NO: 1 (gH), SEQ ID NO: 2 (gL), SEQ ID NO: 3 (UL128), SEQ ID NO: 4 (UL130), and SEQ ID NO: 5 (UL131A), respectively, as long as these mutations are not detrimental to the use of the proteins as antigens, in particular as long as they retain one or more epitopes that can elicit the production of antibodies that can bind to at least a pentameric complex and/or antibodies that can neutralize the biological effects of said pentameric complex. In addition, such mutations should not prevent the capacity of the proteins to form a pentameric complex of the invention. The ability to form a pentameric complex of the invention can be tested by performing protein purification, and analyzing the proteins by e.g. non-reducing PAGE, Western blot and/or size exclusion chromatography. In particular, each protein may comprise a tag which, e.g., may facilitate detection, purification and/or enhances solubility. Exemplary tags which can be used in accordance with the present invention include a His-Tag, a Strep-Tag, a His-Strep-tag, a StrepII-Tag, a Softag 1, a TC-tag, a myc-Tag, a FLAG-tag, a HA-tag, a V5-tag, a Avi-tag, a Calmodulin-tag, a polyglutamate-tag, an amyloid beta-tag, a GST-tag, a MBP-tag or a S-tag, the His-Tag being preferred. The His-Tag may be composed of 6 or 8 His-residues, with 8 His-residues being preferred. The proteins may also be truncated and/or processed into their mature form, for example, the proteins may lack signal sequences present in their native form and/or transmembrane domains.

As said, gH proteins or fragments thereof of the invention can have various degrees of identity to SEQ ID NO: 1 such as at least 60%, 70%, 80%, 85%, 90%, 91%, 92%, 93%, 94%, 95%, 96%, 97%, 98% or 99% identical to the sequence recited in SEQ ID NO: 1. Preferred gH proteins: (i) can dimerise with CMV gL; (ii) form part of the trimeric gH/gL/gO complex; (iii) form part of the pentameric gH/gL/UL128/UL130/UL131Acomplex; (iv) lack a transmembrane domain; and/or (iv) can elicit antibodies in vivo which immunologically cross-react with a CMV virion.

As said, gL proteins or fragments thereof of the invention can have various degrees of identity to SEQ ID NO: 2 such as at least 60%, 70%, 80%, 85%, 90%, 91%, 92%, 93%, 94%, 95%, 96%, 97%, 98% or 99% identical to the sequence recited in SEQ ID NO: 2. Preferred gL proteins: (i) can dimerise with CMV gH; (ii) form part of the trimeric gH/gL/gO complex; (iii) form part of the pentameric gH/gL/UL128/UL130/UL131A complex; and/or (iv) can elicit antibodies in vivo which immunologically cross-react with a CMV virion.

As said, UL128 proteins or fragments thereof of the invention can have various degrees of identity to SEQ ID NO: 3 such as at least 60%, 70%, 75%, 76%, 77%, 78%, 79%, 80%, 81%. 82%, 83%, 84%, 85%, 86%, 87%, 88%, 89%, 90%, 91%, 92%, 93%, 94%, 95%, 96%, 97%, 98% or 99% identical to the sequence recited in SEQ ID NO: 3. Preferred UL128 proteins: (i) can form part of the pentameric gH/gL/UL128/UL130/UL131A complex, and/or (ii) can elicit antibodies in vivo which immunologically cross-react with a CMV virion.

As said, UL130 proteins or fragments thereof of the invention can have various degrees of identity to SEQ ID NO: 4 such as at least 60%, 70%, 80%, 85%, 90%, 91%, 92%, 93%, 94%, 95%, 96%, 97%, 98% or 99% identical to the sequence recited in SEQ ID NO: 4. Preferred pUL130 proteins: (i) can form a pentameric gH/gL/UL128/UL130/UL131 complex; and/or (ii) can elicit antibodies in vivo which immunologically cross-react with a CMV virion.

As said, UL131A proteins or fragments thereof of the invention can have various degrees of identity to SEQ ID NO: 5 such as at least 60%, 70%, 80%, 85%, 90%, 91%, 92%, 93%, 94%, 95%, 96%, 97%, 98% or 99% identical to the sequence recited in SEQ ID NO: 5. Preferred UL131A proteins: (i) can form pentameric gH/gL/pUL128/pUL130/pUL131A complexes, and/or (ii) can elicit antibodies in vivo which immunologically cross-react with a CMV virion.

“Sequence identity” or “% identity” refers to the percentage of residue matches between at least two polypeptide or polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. For purposes of the present invention, the sequence identity between two amino acid sequences or nucleotide is determined using the NCBI BLAST program version 2.2.29 (Jan. 6, 2014) (Altschul et al., Nucleic Acids Res. (1997) 25:3389-3402). Sequence identity of two amino acid sequences can be determined with blastp set at the following parameters: Matrix: BLOSUM62, Word Size: 3; Expect value: 10; Gap cost: Existence=11, Extension=1; Filter=low complexity activated; Filter String: L; Compositional adjustments: Conditional compositional score matrix adjustment. For purposes of the present invention, the sequence identity between two nucleotide sequences is determined using the NCBI BLAST program version 2.2.29 (Jan. 6, 2014) with blastn set at the following exemplary parameters: Word Size: 11; Expect value: 10; Gap costs: Existence=5, Extension=2; Filter=low complexity activated; Match/Mismatch Scores: 2,-3; Filter String: L; m.

The terms “polypeptide” and “protein” are interchangeably used. The term “polypeptide” refers to a protein or peptide that contains two or more amino acids, typically at least 3, preferably at least 20, more preferred at least 30, such as at least 50 amino acids. Accordingly, a polypeptide comprises an amino acid sequence, and, thus, sometimes a polypeptide comprising an amino acid sequence is referred to herein as a “polypeptide comprising a polypeptide sequence”. Thus, herein the term “polypeptide sequence” is interchangeably used with the term “amino acid sequence”.

The term “amino acid” or “aa” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

The invention also provides nucleic acid molecules encoding the gH, gL, UL128, UL130 and/or UL131A proteins or fragments as described herein. These nucleic acid molecules are, e.g., used when expressing one or more of said proteins or are used as nucleic acid molecules as such, e.g., for vaccination. For the purpose of expressing one or more of said proteins, they are cloned into a vector as is commonly known and described herein.

### Co-Expression Step

“Co-expression” is the term used for one or more CMV proteins expressed in a host cell, preferably insect cell or mammalian cell, by using baculovirus, e.g., a Baculovirus expression system or BacMam expression system. An “expression vector” is defined herein as vehicle used to transfer genetic material to a target host cell where the genetic material can be expressed. It is in particular envisaged that the CMV proteins are expressed in a baculovirus expression system. An “expression system” is the combination of an expression vector, and the host cell for the vector that provide a context to allow foreign gene expression in the host cell.

The complex of the present invention may be expressed transiently or stably.

Baculoviruses are rod-shaped double-stranded DNA viruses found mainly in insects.

The baculovirus expression system is typically based on the introduction of a foreign gene into a nonessential viral genome region, e.g. via homologous recombination with a transfer vector containing a target gene. The resulting recombinant baculovirus may lack one of the nonessential genes (e.g. polh, v-cath, chiA) replaced with a foreign gene encoding the heterologous protein which can be expressed in a suitable host cell. These techniques are generally known to those skilled in the art and have been reviewed e.g. by Kosta et al. Nat Biotechnol. 2005; 23(5):567-75. A specific approach for preparing recombinant baculovirus vectors is the Bac-to-Bac® baculovirus system (Invitrogen).

The recombinant baculovirus expression vector of the invention is preferentially capable of replication in a host cell and optionally in a prokaryotic cell such as E. coli. According to the present invention, any baculovirus expression vector derived from a baculovirus commonly used for the recombinant expression of proteins may be used. For example, the baculovirus vector may be derived from, e.g., AcMNPV, Bombyx mori (Bm) NPV, Helicoverpa armigera (Hear) NPV) or Spodoptera exigua (Se) MNPV. The baculovirus vector may be a bacmid.

It is a common prejudice in the prior art that expression of CMV proteins in mammalian cells was preferable because it was known that the produced CMV proteins would have authentic mammalian glycosylation patterns, and thus possessed epitopes that are present on infectious CMV. Accordingly, it was expected, that only such proteins would, when used for immunization, enable generation of antibodies that are able to bind to naturally occurring CMV particles during infection

Surprisingly, the present inventors have found that immunogenic pentameric complexes can be obtained using baculovirus vectors not only from mammalian cells, but also from insect cells. At the same time, using the baculovirus system enables production of the pentameric complex of the invention in high quantities and high purity. Preferably, the pentameric complex produced with the help of the means and methods of the invention exhibits a specific glycosylation pattern, i.e., insect-glycosylation (see Harrison and Jarvis (2006), Adv. Virus Res. 68, 159-191) which renders it unique and thus different from a mammalian-glycosylation.

Thus, the host cell can in general be an insect cell or mammalian cell. Generally, any host cell that is preferably suitable to express nucleic acid molecules to produce the pentameric complex of the invention may be used. The host cell used in accordance with the invention may in particular be an insect cell, preferably Sf9, Sf21, Super Sf9-1 (VE-1), Super Sf9-2 (VE-2), Super Sf9-3 (VE-3), Hi-5, Express Sf+, and S2 Schneider cells, with Super Sf-9-2 being preferred [Oxford Expression Technologies, Cat. No. 600103, Oxford, UK; Fath-Goodin et al. (2006), Adv. Virus Res. 68, 75-90; Kroemer et al. (2006), J. Virol. 80(24), 12291-12228 and US20060134743.]. Exemplary mammalian host cells suitable for use in accordance with the present invention are known in the art and include immortalised cell lines available from the American Type Culture Collection (ATCC) including, but not limited to, HEK293, HEK293F, CHO, HeLa, HUVEC, HUAEC, Huh7, HepG2, BHK, MT-2, Cos-7, Cos-1, C127, 3T3, human foreskin fibroblasts (HFF), bone-marrow fibroblasts, Bowes melanoma, primary neural cells, or epithelial cells. Expression in mammalian cells may cause that proteins produced will have authentic mammalian glycosylation patterns, and thus possess epitopes that are present on infectious CMV particles. Thus, without being bound by theory, production of pentameric complexes of the invention in mammalian cells will lead to the production of antibodies that are able to bind to naturally occurring CMV particles during infection. However, since the present inventors observed that the pentameric complex when produced in insect cells induces neutralizing activity, particularly neutralizing antibodies with ideally block or at least decrease entry of CMV into epithelial/endothelial cells and fibroblasts.

However, and as set out herein, the host cell may also be a mammalian cell. E.g., in the BacMam system, baculovirus expression vectors are used to deliver genes to mammalian cells.

The present inventors have unexpectedly discovered specific parameters which enable, e.g., production of a high yield of the pentameric complexes of the invention. For example, the pentameric complex of the invention may accumulate to a level of more than 1.0 mg per litre of growth medium (e.g. 1, 2, 3, 4, 5, 6, 7, 8, 9, 10, 12, 15, 20, 25, 30, 40, 50, 60, 70, 80, 90 100, 200, 300, 400, 500, 600, 650, 680, 700, 800, 900, 1000) mg per litre of growth medium or more).

In accordance with the foregoing, it is envisaged that the co-expression step involves infecting the host cells with a baculovirus expressing the proteins of the pentameric complex of the invention. It is further envisaged that the baculovirus has a titer of about 10⁷ pfu/mL or higher when infecting said host cell, which preferably has a cell count of about 2*10⁶ cells/mL at infection.

Then, the host cells are cultivated under suitable conditions. Preferably, after 56-65 h post infection, the host cells and/or their supernatant are harvested. In the alternative, host cells and/or their supernatant are harvested when at least 80% (in relation to a total of 100%) of the host cells are viable. Viability of host cells can, for example, be determined by staining cells with trypan blue. Viable cells are not colored, while non-viable cells will be colored. Trypan blue staining can be performed as follows: 1 ml cell suspension is subject to a 0.4% trypan blue stain for about 5 minutes, followed by microscopic observation by preferably using a hemocytometer in order to determine the percentage of viable/non-viable cells in relation to all counted cells (i.e. all counted cells are set to be 100%).

“Harvesting” in all its grammatical forms means the act or process of obtaining the host cells and/or the supernatant, and may for example include trypsinization, filtration, and/or centrifugation. Any method is conceivable as long as the pentameric complexes of the invention can be obtained in their intact or functional form.

It is further envisaged that, in the methods of the present invention, host cells are infected between day 15 and day 50, preferably between day 15 and day 30, preferably at day 18 after thawing and culturing.

In some embodiments, the pentameric complex of the invention is secreted from the cells in which it is expressed. In other embodiments of the invention, the pentameric complex of the invention is not secreted. It is a preferred embodiment that none of the proteins of the pentameric complex contains an additional secretion signal. Without being bound by theory, it is assumed that once the pentameric complex assembles in a host cell, particularly in an insect cell, the gH protein mediates secretion of the entire complex.

### Purification

“Purifying” in all its grammatical forms means removing undesirable compounds, e.g. cells, cell debris, culture medium, baculovirus, either intact or non-intact baculoviruses, etc. Suitable purification methods depending on the expression system, yield, etc. are readily available in the prior art. E.g., purification may include ion exchange chromatography, hydrophobic interaction chromatography, size exclusion chromatography and/or affinity chromatography, all of which have been described extensively before. As said, the purification step includes, inter alia, removing baculoviruses. Such baculoviruses may be contained in the culture medium and/or supernatant obtainable from host cells which were infected with a baculoviral vector or BacMam vector. It is preferred that such baculoviruses be removed when purifying a pentameric complex of the present invention. The present inventors found that in particular ion exchange chromatography, more particularly anion exchange chromatography may be applied to remove baculoviruses from the culture medium and/or supernatant obtainable from a host cell as described herein.

Purifying as used herein also includes that host cells which co-express CMV proteins UL128, UL130, UL131A, gH (UL75) and gL (UL115) may be removed from the culture medium. Said culture medium comprises preferably a pentameric complex of the present invention, since said host cells preferably secrete said pentameric complex. Removing host cells from culture medium may be done by mechanical force, such as by centrifugation or by filtration. Filtration is preferably done by using filtration medium, such as microfiltration filters or on depth-filters. Microfiltration filters may be composed of polyethersulfone or regenerated cellulose. On depth-filters may be composed of polypropylene or glass fibers.

However, it is also envisaged that said host cell do not necessarily have to secrete said pentameric complex. If so, then said host cells may be harvested. After harvest, said host cells may be broken up, e.g., enzymatically or mechanically in order to release a pentameric complex which may then be purified as described herein.

After purification, it is preferred that a chelating agent such as EDTA or EGTA is added to the complex. Preferably EDTA is present at a final concentration of 20 mM. Subsequently, it is preferred that the 20 mM final concentration of EDTA is reduced by dialysis to 3 mM final concentration or lower as described herein.

### Storage

“Storing” in all its grammatical forms means preserving (for future use), preferably under conditions which maintain the pentameric complex of the invention in its intact or functional form, i.e. the pentameric complex preferably resembles its naturally occurring form and/or is able to induce neutralizing antibodies. It is thus envisaged that storing conditions do not promote (or do even prevent) disintegration of the pentameric complex of the invention. The term “disintegration” is to be understood in its broadest sense herein and can mean “disassembly” and/or “denaturation”. Storage of the pentameric complex of the invention is envisaged in a buffer solution comprising a chelating agent and/or a stabilizing agent.

In general, any chelating agent and/or stabilizing agent is suitable as long as it enables storage of the pentameric complex of the invention and does not promote its disintegration. An exemplary useful chelating agent in the context of the present invention is EDTA. EDTA can be present in said buffer solution at a concentration of 20 mM or less, such as 15 mM, 10 mM, 9 mM, 8 mM, 7 mM, 6 mM, 5 mM, 4 mM, 3 mM, 2 mM, or 1 mM. In particular, EDTA can be present at a concentration of 3 mM or less. Exemplary stabilizing agents for use in accordance with the present invention include glycol, arginine, sorbitol, glycerol and/or sucrose. In particular, the buffer solution may comprise 100-200 mM arginine, 100 mM sorbitol, 20% glycerol (w/v), 20% sucrose (w/v), 0.5% NP-40, 0.2% Brij-35 and 0.5% Chaps.

The buffer solution in accordance with the present invention may comprise Tris buffer, NaCl, KCl and have a pH of 6.5. In particular, the buffer solution may comprise 25 mM Tris buffer, 150 mM NaCl, 3 mM KCl. The buffer solution may also be a sodium/potassium phosphate buffer. Said buffer may have a pH between 6.0 and 7.0.

### Vector Design

The present invention provides one or more vectors comprising open reading frames (ORFs) encoding CMV proteins UL128, UL130, UL131, gH and gL.

The vector can contain elements for propagation in bacteria (e.g. E. coli), yeast (e.g. S. cerevisiae), insect cells and/or mammalian cells. Preferably, said vector is a Baculovirus vector or a Baculovirus BacMam vector.

In the BacMam system, baculovirus vectors are used to deliver genes into mammalian cells. The BacMam system can be used for gene delivery to a broad range of cell lines and primary cells as host cells, an exemplary list of which is included elsewhere herein. The unmodified baculovirus is able to enter mammalian cells, however its genes are not expressed unless a mammalian recognizable promoter is incorporated upstream of a gene of interest. Thus, it is envisaged that the BacMam vector of the invention comprises a mammalian promoter upstream the genes encoding the proteins of the pentameric complex of the invention. The vector may comprise additional elements as described elsewehere herein, e.g. antibiotic resistance genes, elements for propagation in E. coli, S. cerevisiae etc.

In said baculovirus vector the v-cath and/or ChiA gene can be functionally disrupted.

Generally, the open reading frames (ORFs) encoding CMV proteins UL128, UL130, UL131A, gH and gL of the pentameric complex of the invention can be present on one or more vectors, e.g. on two vectors. Accordingly, one, two, three, or four of the ORFS are on a first vector, while the remaining ORF/ORFs are on a second vector. Preferably, however, said ORFs are present on a single vector. ORFs may also be present in polygenic form (EP1945773).

The ORFs can for example be located in the following order from 5′ to 3′ in said vector:

(i) gH, gL, UL128, UL130, UL131A;

or (ii) gL, UL128, UL130, UL131A, gH.

In particular, it is envisaged that
(a) in (i) the gH ORF is transcribed in 3′ direction, the gL ORF is transcribed in 5′ direction, the UL128 ORF is transcribed in 3′ direction, the UL130 ORF is transcribed in 3′ direction, and the UL131A ORF is transcribed in 3′ direction;
(b) in (i) gH ORF is transcribed in 3′ direction, the gL ORF is transcribed in 3′ direction, the UL128 ORF is transcribed in 3′ direction, the UL130 ORF is transcribed in 3′ direction, and the UL131A ORF is transcribed in 3′ direction;
(c) in (ii) gL ORF is transcribed in 5′ direction, the UL128 ORF is transcribed in 3′ direction, the UL130 ORF is transcribed in 3′ direction, the UL131A ORF is transcribed in 3′ direction, and the gH ORF is transcribed in 3′ direction.

Additional examples of vectors are the following vectors (pRBT136-x) for the generation of the pentameric complex of CMV (the construction and processing of said vectors is described in Example 1 of PCT/EP2013/072717) and are thus preferred vectors that can be applied for the expression of a pentameric complex of the present invention. Accordingly, the vectors listed in Table 1 below are preferred exemplary vectors of the vector that is used for the co-expression of one or more of the proteins of the pentameric complex. Notably, independent of the specific nucleotide sequence shown in the left column of the below Table, the arrangement of the genes encoding the proteins of the pentameric complex as shown in Table 1 below are also preferred arrangements that are an embodiment of the present invention.

Abbreviations: c: consensus sequence; H: His-tag; SH: Streptavidin-His-tag; V: VR1814, pcl: precission protease, pcll: precission and TEV protease, DT: dimerization tool. For the description of the genes in Table 1 the shorter CMV nomenclature (gB, gH, gL, gO as well as “UL” without prefix and UL48 without suffix “A”) is used.

TABLE 1

| Vector variant | | Variant with |
|---|---|---|
| (pRBT136-x) | | dimerisation |
| SEQ ID | Contained genes | tool (DT) at gH |
| 6 | gH-gL-UL128-UL130-UL131A, | gH (DT; Towne) |
| | Towne | |
| 7 | gH-gL-UL128(c)-UL130- | gH (DT; Towne) |
| | UL131A, Towne | |
| 8 | gH-gL(H)-UL128-UL130- | |
| | UL131A, Towne | |
| 9 | gH-gL(H)-UL128(c)-UL130- | gH (DT; Towne) |
| | UL131A, Towne | |
| 10 | gH-gL(SH)-UL128-UL130- | |
| | UL131A, Towne | |
| 11 | gH-gL(SH)-UL128(c)-UL130- | gH (DT; Towne) |
| | UL131A, Towne | |
| 12 | gH-gL-UL128(Towne)- | |
| | UL130(V)-UL131A(V) | |
| 13 | gH-gL-UL128(c)-UL130(V)- | |
| | UL131A(V) | |
| 14 | gH(DT)-gL-UL128-UL130- | |
| | UL131A, Towne | |
| 15 | gH (DT)-gL-UL128(c)- | |
| | UL130-UL131A, Towne | |
| 16 | gH(DT)-gL(SH)-UL128- | |
| | UL130-UL131A, Towne | |
| 17 | gH (DT)-gL(SH)-UL128(c)- | |
| | UL130-UL131A, Towne | |
| 18 | gH (without membrane | |
| | anchor, His)-gL-UL128- | |
| | UL130-UL131A, Towne | |
| 19 | gH (without membrane | |
| | anchor, SH)-gL-UL128- | |
| | UL130-UL131A, Towne | |
| 20 | gH (without membrane | |
| | anchor, H-pcI)-gL- | |
| | UL128-UL130-UL131A, Towne | |
| 21 | gH (without membrane | |
| | anchor, H- pcII)-gL- | |
| | UL128-UL130-UL131A, Towne | |
| 67 | gL-UL128c-UL130V-UL131AV- | |
| | gH (without membrane | |
| | anchor, H), Towne | |
| 68 | gLT-UL128T-UL130V-UL131AV- | |
| | gH (without membrane | |
| | anchor, H), Towne | |
| 69 | gL-UL128c-UL130V-UL131AV- | |
| | 2a-gH (without membrane | |
| | anchor, H), Towne | |
| 70 | gL-UL128-UL130V-UL131AV-2a- | |
| | gH (without membrane anchor, | |
| | H), Towne | |
| 71 | gL-UL128c-2a-UL130V-2a- | |
| | UL131AV-2a-gH (without | |
| | membrane anchor, H), Towne | |
| 72 | gL-UL128c-2a-UL130V-2a- | |
| | UL131AV-2a-gH (without | |
| | membrane anchor, H), Towne | |

The vector backbone pRBT136 used preferably for this invention contains an origin of replication for E. coli, e.g. pBR322ori, and yeast, e.g. 2 micron ori, the polh and p10 promoters for expression in insect cells, the terminators SV40 and HSVtk, several resistance markers (ampicillin, gentamycin), a yeast selection marker (URA3), transposon sites (Tn) and a multiple cloning site (MCS).

By way of example, an expression cassette containing promoter—gene of interest—terminator is PCR amplified at the 5′ site with a 35-40 nt overhang at the 5′ site, and at the 3′ site with a further and different 35-40 nt overhang. For homologous recombination with a second expression cassette, having the same organization, the PCR product contains, at the 5′ site, the complementary sequence of the 35-40 nt overhang to the 3′ site of the previous PCR product. The remaining overhangs at the 5′ site of the first PCR product and the 3′ site of the second PCR product are homologous to the 3′ and the 5′ end of a linearized vector (pRBT136), respectively. The homologous recombinations in a sequence are then conducted in yeast, preferably in Saccharomyces cerevisiae. The number of the expression cassettes/PCR products to be assembled in parallel with the strategy described before is increased according to the needed number of genes to be assembled. By this means multiple genes/expression cassettes are assembled in parallel. The assembled genes are flanked by the transposon sites. These are used for transposition of the genes into the baculovirus genome. The resulting baculovirus co-expression vector ensures that the genes are co-expressed from the same single cell. Yield and product composition vary dependent on the number of proteins and production parameters. The production parameters such as cell line, cell count at infection (CCI), amount of recombinant virus inoculum (multiplicity of infection, MOD and time of harvest (TOH) are determined in respect to yield and early harvest using a matrix system and a small scale production system (2-20 ml; Ries, C., John C., Eibl R. (2011), A new scale down approach for the rapid development of Sf21/BEVS based processes—a case study. In Eibl R., Eibl D. (Editors): Single-use technology in Biopharmaceutical Manufacture, 207-213, John Wiley & Sons, Hoboken, N.J.). The defined parameters are then used to produce the respective product at larger scale. The pentameric complex of the invention is manufactured using modern disposable tissue culture techniques which allow for high production capacity.

The vector may contain one or more further elements, including, e.g., an origin of replication, promoters, cloning sites, genetic markers, antibiotic resistance genes, epitopes, reporter genes, targeting sequences and/or protein purification tags. The person skilled in the art will readily know which elements are appropriate for a specific expression system.

In particular, the vector in accordance with the invention may further contain elements for propagation in bacteria (E. coli), yeast (S. cerevisiae), insect cells and/or mammalian cells, such as origin of replication, selection markers, etc.

It is envisaged that the vector comprises a promoter for gene expression. Each of the ORFs described herein is driven by a promoter. The promoters are preferably selected from the group consisting of polh, p10 and p_{XIV} very late baculoviral promoters, vp39 baculoviral late promoter, vp39polh baculoviral late/very late hybrid promoter, pca/polh, pcna, etl, p35, egt, da26 baculoviral early promoters; CMV-IE1, UBc. EF-1, RSVLTR, MT, Simian virus 40 promoter, CAG promoter (beta-actin promoter with CMV-IE1 enhancer), hepatitis B virus promoter/enhancer, human ubiquitin C promoter, hybrid neuronal promoter, p_{DS47}, Ac5, and P_{GAL} and P_{ADH}. Each of the ORFs described herein is followed by a terminator sequence such as HSVtk terminator, SV40 terminator, or bovine growth hormone (BGH) terminator.

One or more of the proteins of the pentameric complex of the invention may comprise PreScission protease or PreScission and TEV protease. It is in particular envisaged that, e.g, the gH and/or gL protein may comprise PreScission protease or PreScission and TEV protease.

It is further envisaged that the baculovirus v-cath and/or ChiA activity can be functionally disrupted, which means that preferably no functional v-cath and/or ChiA is present and/or expressed Most baculoviruses encode a chitinase (chiA) and a viral cathepsin-like protease (v-cath) which are retained in cells and released upon virus-induced lysis to liquefy host carcasses at the end of the infection (Hawtin, R E et al. Virology. 1997; 238, 243-253). These genes are non-essential for baculovirus replication and can, thus, be inactivated, e.g. by partial or full deletion, insertion mutagenesis or by one or more inactivating point mutations.

The pentameric complex of the invention can be prepared at various levels of purity e.g. at least 80%, 85%, 90%, 95%, or 99% of total protein by mass, e.g. as determined by gel electrophoresis. These high levels of purity make the complexes suitable for, e.g., use as an immunogen in diagnostic applications or as an antigen in immunogenic formulations. Such level of purity is preferably obtainable by (i) removing host cells from the culture medium as described herein, (ii) applying chromatography as described herein, e.g. applying affinity chromatography, if a tagged-version of the pentameric complex is expressed by host cells, followed by (iii) removing baculoviruses, if a baculovirus expression system is used, through ion exchange chromatography, in particular anion exchange chromatography. Any of these steps may be repeated, with step (ii) being preferably repeated as last step. These steps may be in the order (i), (ii) and (iii); (ii), (i) and (iii); (i), (iii) and (ii); (iii), (ii) and (i); (ii), (iii) and (i); or (iii), (i) and (ii). Preferably, step (ii) is repeated as last step.

### Composition

The pentameric complex of the invention can also be in the form of a composition. The composition of the invention may further comprise buffers, reducing agents, stabilizing agents, chelating agents, bulking agents, osmotic balancing agents (tonicity agents); surfactants, polyols, anti-oxidants; lyoprotectants; anti-foaming agents; preservatives; and colorants, detergents, sodium salts, and/or antimicrobials etc. The composition may be free from polyacrylamide.

In some embodiments, the composition does not contain polyacrylamide. In some embodiments, the composition is a liquid e.g. an aqueous liquid, not a gel. In some embodiments, the protein complex is not immobilized within the composition. For example, said pentameric complex may not be present in a gel, or on a film, membrane, paper or slide.

A composition may be sterile and/or pyrogen-free. Compositions may be isotonic with respect to humans.

### Neutralizing Activity

The inventors have discovered that the pentameric complexes of the invention are able to induce an immunogenic response. The term “immunogenic response” means “adaptive immune response”, and in general includes humoral and/or cell-mediated immune responses, preferably in vivo. Preferably, the immunogenic response involves the induction of neutralizing activity, whereby the neutralizing activity is preferably in the form of neutralizing antibodies.

A “neutralizing antibody” is an antibody that can neutralize (abolish or decrease) the biological effect of an antigen, e.g. the ability of a pathogen to initiate and/or perpetuate an infection in a host. Preferably, the neutralizing antibodies generated in response to the pentameric protein complex of the invention cross-react with CMV virion particles, thereby conferring immunity against CMV infection. Without being bound by theory, it is believed that the neutralizing antibodies generated in response to the pentameric protein complex of the invention abolish, or at least decrease the ability of CMV virion particles to enter epithelial, endothelial (Epi/EC) and/or fibroblast cells, the “and” combination being preferred. It is envisaged that the efficiency of entry into cells is decreased by at least about 25, 30, 35, 40, 45, 50, 55, 60, 65, 70, 75, 80, 85, 90, 95, 99 or 100%, as determined by standardized tests that are known to those of skill in the art. As used herein, “efficiency” is defined as the number of cells infected in the presence of antibody as a percentage of the number infected in the absence of antibody. Neutralization assays for screening/identifying/determining in particular neutralizing antibodies can be done as described in the legend of FIG. 3 and in Example 3.

It is thus envisaged that the pentameric complexes of the invention may be able to induce immunity against CMV infection. These two functions (i.e., induction of an immunogenic response and induction of immunity) are dependent on the retention of epitopes on the pentameric complexes of the invention that can elicit the production of antibodies, including neutralizing antibodies. A range of conformational epitopes for the pentameric complex are known; see Macagno (2010), Journal of Virology 84 (2010): 1005-13.

In view of the foregoing, the present invention provides a pentameric complex which is preferably capable of inducing neutralization activity that inhibits both epithelial/endothelial (Epi/EC) and fibroblast infection.

### Method for Production

In a second aspect, the present invention also provides a method for the production of a pentameric complex composed of CMV proteins UL128, UL130, UL131A, gH (UL75) and gL (UL115), comprising
(i) co-expressing baculovirus CMV proteins UL128, UL130, UL131A, gH (UL75) and gL (UL115) in a host cell;
(ii) purifying the pentameric complex from host cells and/or supernatant obtained from said co-expression; and
(iii) optionally storing the purified pentameric complex in a buffer solution comprising a chelating agent and/or a stabilizing agent.

It is to be noted that the embodiments described in the context of the pentameric complex of the invention also apply to the method of the invention, mutatis mutandis.

### Pharmaceutical/Vaccine Composition

In a third aspect, a pharmaceutical composition or immunogenic composition comprising a therapeutically effective amount of the pentameric complex of the invention or obtainable by the method of the invention and optionally a pharmaceutically acceptable carrier or adjuvant is provided. The pharmaceutical composition or immunogenic composition of the present invention may also comprise a vector as described herein.

A “therapeutically effective amount” is an amount sufficient to elicit a desired therapeutic effect. E.g. in a vaccine composition the therapeutically effective amount may be the amount sufficient to induce an immune response, e.g. the production of neutralizing antibodies in a subject. The subject can be preferably a mammal, which can be, for instance, a mouse, rat, guinea pig, hamster, rabbit, dog, cat, or primate. Preferably, the subject is a human.

The term “pharmaceutically acceptable” may in particular mean approved by a regulatory agency or other generally recognized pharmacopoeia for use in animals, and more particularly in humans.

The pharmaceutical/immunogenic composition of the invention is envisaged for therapeutic treatment of a subject. The term “therapeutic treatment” in all its grammatical forms includes therapeutic or prophylactic treatment. A “therapeutic or prophylactic treatment” comprises prophylactic treatments aimed at the complete prevention of clinical and/or pathological manifestations or therapeutic treatment aimed at amelioration or remission of clinical and/or pathological manifestations.

To reduce the chance of congenital disease a prophylactic vaccine to prevent the first CMV infection of the mother is desirable, whereas an effective therapy is needed in the case a mother is diagnosed with an active CMV infection. A pentameric complex of the present invention is particularly envisaged to be applied as a prophylactic vaccine, e.g. for expectant mothers, children or transplant patients before transplantation.

The immunogenic composition can further comprise gB protein, gM protein, pp65 protein, IE-1 protein, dimer of gL/gH protein, dimer of gM/gN protein, trimer of gL/gH/gO, virus-like particles (VLPs) comprising one or more capsid or capsid precursor proteins, one or more surface proteins from CMV, and/or or one or more tegument proteins. “gB” is used interchangeably with UL55 herein. “gM” is used interchangeably with UL100 herein. “pp65” is used interchangeably with UL83 herein. “IE-1” is used interchangeably with UL123 herein. “gO” is used interchangeably with UL74 herein. It is to be understood that the aforementioned proteins are not restricted with respect to a specific sequence. Mutated and truncated forms of the aforementioned proteins are also envisaged and encompassed in the immunogenic composition of the present invention. The proteins can also be present in the immunogenic composition of the invention in modified form. Exemplary modifications have been described in the context of the proteins of the pentameric complex of the invention and are also applicable to gB, gM, pp65, IE-1 and gO.

The vaccine or vector vaccine, respectively, of the invention may further comprise a soluble form of complement receptor type 1 (sCR1).

A “virus-like particle (VLP)” is a complex of viral structural proteins (e.g., surface proteins), which resembles a virus, but does not contain any viral genetic material, and is therefore non-infectious. VLPs used in accordance with the present invention can in principle comprise any viral proteins to which an immune response is desired to be elicited. E.g., the VLPs used in accordance with the present invention may comprise CMV capsid or capsid precursor proteins, surface proteins and/or tegument proteins, B-cell and/or T-cell epitopes, and/or proteins selected from the group of additional foreign antigenic sequences, cytokines, CpG motifs, g-CMSF, CD19 and CD40 ligand and/or fluorescent proteins, proteins useful for purification purposes of the particles or for attaching a label, and/or proteinaceous structures required for transport processes.

Further the immunogenic composition can comprise a nucleic acid molecule encoding gB, gM, pp65, IE-1 or IE-2. E.g., the nucleic acid can be DNA or RNA. E.g., the nucleic acid can be in form of a vector or a plasmid. Complexed and stabilized forms are also envisaged.

The present invention also provides an immunogenic composition comprising a vector encoding a pentameric complex of the invention or any other CMC complex as described herein. Said vector can be DNA- or RNA-based. Suitable vectors for use in accordance with the immunogenic composition include DNA-based vectors such as baculovirus vectors, BacMam vectors, adenovirus vectors, lentiviral vectors, AAV vectors, herpesvirus vectors, poxvirus vectors, and Eppstein-Barr virus (EBV) vectors. The use of naked DNA; e.g. in the form of a plasmid, and optionally complexed and/or in stabilized form (e.g. lipoplexes, polyplexes, dendrimers, virosomes and complexes with inorganic nanoparticles) is also envisaged. Suitable RNA-based vectors include retroviral vectors, Semliki forest virus (SFV), Sindbis virus (SIN) and Venezuelan equine encephalitis virus (VEE) vectors.

The immunogenic compositions of the present invention can further comprise a modified vaccinia virus Ankara (MVA) comprising one or more proteins of the CMV pentameric complex composed of UL128, UL130, UL131, gH (UL75) and gL (UL115).

Furthermore, the present invention provides an immunogenic composition for use in a method of vaccinating a subject against CMV, comprising administering as priming composition said immunogenic composition and as boosting composition a (i) gH/gL dimer, (ii) a UL130/UL131A-dimer, (iii) gM/gN dimer (iv) a gH/gL/UL128/UL130/UL131A-pentamer, (v) gB, (vi) gM, (vii) pp65, (viii) IE-1, (ix) IE-2, (x) a modified vaccinia virus Ankara (MVA) comprising one or more proteins of the CMV pentameric complex composed of UL128, UL130, UL131, gH (UL75) and gL (UL115), (xi) a modified vaccinia virus Ankara (MVA) comprising gB, a gH/gL dimer, pp65 protein or IE-1 protein, (xii) virus-like particles (VLPs) comprising one or more capsid or capsid precursor proteins, one or more surface proteins from CMV, or one or more tegument proteins, (xiii) nucleic acid sequence encoding any one of the compounds as defined in (i) to (xii), (xiv) peptides from flagellin, (xv) CpG motifs, and/or (xvi) LCMV to said subject.

Said boosting composition can also be used as priming composition and said priming composition is used as boosting composition.

In the prime boost regimen, a prime/boost vaccine is used which is composed of two or more types of vaccine including a vaccine used in primary immunization (prime or priming) and a vaccine used in booster immunization (boost or boosting). Usually, the vaccine used in primary immunization and the vaccine used in booster immunization are different from each other. Primary immunization and boosting immunization may be performed sequentially, this is, however, not mandatory. The prime/boost regimen includes, without limitation, e.g. DNA prime/protein boost, DNA prime/viral vector boost (e.g. using MVA).

### Carrier

The terms “carrier” and “excipient” are used interchangeably herein. Pharmaceutically acceptable excipients include, but are not limited to diluents (fillers, bulking agents, e.g. lactose, microcrystalline cellulose), disintegrants (e.g. sodium starch glycolate, croscarmellose sodium), binders (e.g. PVP, HPMC), lubricants (e.g. magnesium stearate), glidants (e.g. colloidal SiO₂), solvents/co-solvents (e.g. aqueous vehicle, Propylene glycol, glycerol), buffering agents (e.g. citrate, gluconates, lactates), preservatives (e.g. Na benzoate, parabens (Me, Pr and Bu), BKC), anti-oxidants (e.g. BHT, BHA, Ascorbic acid), wetting agents (e.g. polysorbates, sorbitan esters), anti-foaming agents (e.g. Simethicone), thickening agents (e.g. methylcellulose or hydroxyethylcellulose), sweetening agents (e.g. sorbitol, saccharin, aspartame, acesulfame), flavouring agents (e.g. peppermint, lemon oils, butterscotch, etc), humectants (e.g. propylene, glycol, glycerol, sorbitol). The person skilled in the art will readily be able to choose suitable pharmaceutically acceptable excipients, depending, e.g., on the formulation and administration route of the pharmaceutical composition.

A non-exhaustive list of exemplary pharmaceutically acceptable excipients includes (biodegradable) liposomes; microspheres made of the biodegradable polymer poly(D,L)-lactic-coglycolic acid (PLGA), albumin microspheres; synthetic polymers (soluble); nanofibers, protein-DNA complexes; protein conjugates; erythrocytes; or virosomes. Various carrier based dosage forms comprise solid lipid nanoparticles (SLNs), polymeric nanoparticles, ceramic nanoparticles, hydrogel nanoparticles, copolymerized peptide nanoparticles, nanocrystals and nanosuspensions, nanocrystals, nanotubes and nanowires, functionalized nanocarriers, nanospheres, nanocapsules, liposomes, lipid emulsions, lipid microtubules/microcylinders, lipid microbubbles, lipospheres, lipopolyplexes, inverse lipid micelles, dendrimers, ethosomes, multicomposite ultrathin capsules, aquasomes, pharmacosomes, colloidosomes, niosomes, discomes, proniosomes, microspheres, microemulsions and polymeric micelles. Other suitable pharmaceutically acceptable excipients are inter alia described in Remington's Pharmaceutical Sciences, 15^{th} Ed., Mack Publishing Co., New Jersey (1991) and Bauer et al., Pharmazeutische Technologie, 5^{th} Ed., Govi-Verlag Frankfurt (1997).

### Adjuvant

The pharmaceutical or immunogenic composition of the invention may further comprise an adjuvant to stimulate the immune system's response to the pentameric complex. Exemplary adjuvants for use in accordance with the present invention include inorganic compounds such as alum, aluminum hydroxide, aluminum phosphate, calcium phosphate hydroxide, mineral oils, such as paraffin oil, virosomes, bacterial products, such as killed bacteria Bordetella pertussis, Mycobacterium bovis, toxoids, nonbacterial organics, such as squalene, thimerosal, detergents (Quil A), cytokines, such as IL-1, IL-2, IL-10 and IL-12, and complex compositions such as Freund's complete adjuvant, and Freund's incomplete adjuvant. Generally, the adjuvant used in accordance with the present invention preferably potentiates the immune response to the pentameric complex of the invention and/or modulates it towards the desired immune responses.

A variety of routes are applicable for administration of the pharmaceutical or immunogenic composition of the present invention, including, but not limited to, orally, topically, transdermally, subcutaneously, intravenously, intraperitoneally, intramuscularly or intraocularly. However, any other route may readily be chosen by the person skilled in the art if desired.

The exact dose of the pharmaceutical/immunogenic composition of the invention which is administered to a subject in need thereof will depend on the purpose of the treatment (e.g. treatment of acute disease vs. prophylactic vaccination). Adjustments for route of administration, age, body weight, general health, sex, diet, time of administration, drug interaction and the severity of the condition may be necessary, and will be ascertainable with routine experimentation by those skilled in the art.

### Source

In a fourth aspect, in the pentameric complex of the invention, at least one, two, three or four of said proteins can be from a CMV strain other than the CMV strain from which the remaining proteins are from.

A “strain” in accordance with the present invention refers to a CMV genotype variant. The expression “from a CMV strain” is used interchangeably with the expression “derived from a CMV strain” herein and is to be understood in its broadest sense herein as being “retraceable to” a CMV strain, i.e. as having a naturally occurring counterpart or ancestor in a CMV strain. The term also encompasses modified proteins, e.g. having mutations such as amino acid deletions, insertions or conversions with respect to their naturally-occurring counterparts, or tagged proteins.

For example, the CMV proteins can be derived from CMV strain Towne, Towne having the genome as deposited with NCBI GenBank under accession number FJ616285.1, Toledo (GU937742.1), AD169 (FJ527563), Merlin (AY446894.2), TB20/E (KF297339.1), VR1814 (GU179289). The aa Y at position 204 was exchanged by the aa F, according to Patrone et al., J. Virol., 2005, 79, 8361-8373 the major problem of expression of UL130 is the frameshift at the same position leading to an amino acid expansion to the next ORF. Notably, CMV strain Towne (ACCN: FJ616285.1) itself does not express a functional pentameric complex; however, the present inventors have surprisingly found that a functional pentameric complex can be obtained from Towne when using the nucleotide sequence from Towne as deposited with GenBank under accession number FJ616285.1 for gene synthesis (ORFs UL75 (protein ID: ACM48053.1), UL115 (ACM48085.1), UL128 (AAR31451.1), UL130, UL131A (AAR31453.1)). Such a pentameric complex elicited generation of neutralizing antibodies in mice, which were able to prevent CMV entry into fibroblast cells. Thus, the ORFs encoding UL75, UL115, UL128, UL130, UL131A of CMV Towne strain as deposited with GenBank under accession number FJ616285.1 are preferred. Likewise, the proteins encoded by said ORFs are preferred.

In addition, the present inventors have found that it is also possible to use the ORFs present in the CMV strain Towne having at position 204 of the amino acid sequence of the UL130 ORF the amino acid F (Phe) as well as the repair of frameshift at the same position of the labstrain Towne (grown on human foreskin fibroblasts) resulting in a functional amino acid Y (Tyr). Using the modified pUL130 still provides a functional pentameric complex.

### Virus

In a fifth aspect, the invention provides a modified CMV Towne strain having the genome as deposited with NCBI GenBank under accession number FJ616285.1 and having at position 204 of the amino acid sequence of the UL130 ORF the amino acid F as well as the repair of frameshift at the same position of the labstrain Towne (grown on human foreskin fibroblasts) resulting in an functional aa Y.

A better understanding of the present invention and of its advantages will be had from the following examples, offered for illustrative purposes only. The examples are not intended to limit the scope of the present invention in any way.

### EXAMPLES

### Example 1

### Expression, Purification and Characterization of the Pentameric CMV Complex

According to the protein expression parameters defined based on systematic optimization (see Example 1 of PCT/EP2013/072717) the pentameric CMV complex comprising gpUL75 (gH-His)-gpUL115 (gL)-gpUL128-gpUL130-gpUL131A is produced in disposable 2 L shake flasks (culture volume 700 ml) in fall army worm Spodoptera frugiperda cells (Sf9) by co-expression from a single baculovirus (SEQ ID NO:18). The production parameters were as follows: Initial cell count at infection (CCI) of 2×10⁶ cells/ml, a multiplicity of infection (MOI) of 0.25 pfu/ml, incubation at 27° C. at 100 rpm. Harvest took place at day 3 post infection (p.i.) at a viability around 80%. The production was controlled by daily sampling, determining cell count and viability. The complex containing supernatant was loaded on 2×5 ml HisTrap colums (GE Healthcare). The complex was purified using a linear gradient from zero to 500 mM imidazole over 50 column volumes (CV, equivalent to 500 ml. The different chromatographic fractions were analysed by biochemical methods. 150 μl of different fractions were precipitated with acetone, resuspended in 30 μl 20 mM Tris, 150 mM NaCl buffer, pH 7.4. For loading onto a 4-12% Bis-Tris NuPAGE gel (Invitrogen) 4× loading dye was added according to the manufacturer's protocol, followed by the electrophoreses for 15 min at 150 V and for 45 min at 180 V using MOPS running buffer. The gels were stained over night with SimplyBlue SafeStain reagent (Invitrogen) and destained with water. For concentration and further purification a 2^{nd} IMAC chromatography was done using a 1 ml HisTrap column by a linear gradient over 50 column volumes from 0-500 mM imidazole. The different fractions were analysed like the 1^{st} IMAC step by Coomassie stained SDS-PAGE and immunoblotting using an anti-His antibody. All complex containing fractions were pooled, concentrated to a final volume of 5 ml using a 50 kDa cut off Amicon filter unit (Millipore) and loaded for a final purification step on a size exclusion column (XK16/69, Superdex200 pg) and analysed by SDS-PAGE followed by Coomassie staining and immunoblotting using an anti-His antibody. Total protein is determined via a Bradford assay adapted to a 96-well plate format (BCA, Pierce). 20 μl of unknown or standard sample was diluted in 180 μl of buffer. Serial 2-fold dilutions are made to the standard in triplicate (pure bovine serum albumin) or unknown samples. 100 μl of 2× stock Bradford reagent (Pierce) was added per well. The plate is then mixed and absorbance was measured at 595 nm in a microtiter plate reader. The pentameric CMV complex contained in the different fractions from the chromatographic based purification is further analysed by investigating the binding of a specific antibody against the His-tag added to gpUL75 (gH, mouse-anti-His, AbD Serotec) and the gpUL75 itself (gH, mouse-anti-gH, Santa Cruz). Therefore the different samples were 1:10 diluted in 100 μl/well coating buffer (0.1 M Na₂HPO₄, pH 9) in a 96-well pre-absorbed ELISA plate and incubated over night at 4° C. Afterwards the plate was washed 3× with 195 μl/well wash buffer (1×PBS, 0.05% Tween 20) followed by a 1 h blocking at room temperature with 195 μl/well 3% BSA in 1×PBS solution. After 3 washing steps the specific antibody, the anti-His antibody (anti-His) as well as the anti-gpUL75 (anti-gH) in a concentration of 1 pg/ml in 3% BSA, 1×PBS, 0.05% Tween 20, pH 7 was added (100 μl/well) and incubated for 1 h at room temperature followed by further 3 wash steps. For detection a 1 h incubation with the appropriate secondary antibody (anti-mouse-IgG-HRP, 1:1000 dilution in 3% BSA, 1×PBS, 0.05% Tween20, pH 7) was conducted. The binding of the specific antibody to the pentameric complex was detected using 100 μl/well TMP substrate reagent (BD Biosciences, San Diego, USA; according to manufacturer's protocol), wherefore the reaction is stopped after 3-15 min with 100 μl 1 M HCl, followed by OD measurement at 450 nm in a microplate reader. Proteins of the pentameric complex were identified by mass spectroscopy with high coverage and the molecular weight of the proteins of the pentameric complex was determined as follows: UL128 (MW: 19702.982), UL130 (MW: 24618.466, UL131A (MW: 14865.502), gL 8MW: 30894.892), gH (MW: 83203.292).

Exemplary expression vectors (pRBT136-x) for the generation of the pentameric CMV complex are illustrated in Table 1, above.

After expression and purification, the pentameric complex may be admixed with, e.g., a chelating agent and/or stabilizing agent as described herein.

### Example 2

### In Vivo Study in Mice

For the in vivo study Balb/C mice were used in a prime-boost-boost regimen. Each group contained 8 mice and each mouse received 20 pg protein per injection. Pre-immune sera were taken after 14 days quarantine of the mice (day 0). The first injection took place 10 days later followed by a booster injection at day 42. The first bleeding was done at day 49. The 2^{nd} booster injection was performed at day 61 followed by a further bleeding at day 70. The final bleeding took place at day 85 followed by the investigation of humoral and cellular immune response.

### Example 3

### Humoral Immune Response Based on Neutralization Assay

The humoral immune response of the vaccine candidate based on the pentameric complex (SEQ ID: 18) was investigated by a neutralization assay of the mice sera from example 2 in comparison to sera from CMV negative and CMV positive human blood donors. A BAC (bacterial artificial chromosome)-reconstituted VR1814 strain carrying a GFP molecule for analytical reasons (fix-EGFP) was used for the infection of fibroblasts (MRC-5) and epithelial (ARPE-19) cells to visualize the neutralisation potential of the mouse sera from Example 9. 2×10⁴ cells/well were seeded into a 96 well plate in RPMI medium containing 10% FCS (fetal calf serum). A serum pool of the 8 mice was generated and added in 2-fold serial dilutions (1:20 to 1:2560) in 100 μl/well RPMI/FCS medium. The above mentioned fix-EGFP VR1814 virus was added in a tissue culture infectious dose (TCID) of 1000 virus molecules/well which was determined in a pre-assay. The 96 well plates were incubated for 8 days at 37° C. in a CO₂ controlled atmosphere. The determination of green cell was performed in a plate reader with the following parameters: fluorescein-filter [excitation 485/20, emission 530/25), bottom reading mode, time: 0.1 sec, 25× measurements/well after 96 h incubation. The neutralization potency was determined as the dilution of the sera able to show a 50% virus infection inhibition. The following controls were performed: cell control (cells+PBS), virus control (only infected cells) and 5 CMV positive and 5 CMV negative sera from human blood donors.

### Example 4

### Cellular Immune Response Based on EliSpot Data (Multiplex Assay)

At day 85 of the in vivo study (Example 2) mice were killed and the spleenocytes prepared for the analysis of 10 different cytokines. For the restimulation of the spleenocytes the complex as well as mixes of synthetic peptides were used. The following proteins were verified: HIVgag, pUL83, gpUL75, gpUL115, gpUL55, gpUL128, gpUL130 and gpUL131A. An epitope prediction of each protein was done using several bioinformatics algorithms. For each protein a mix of 4 peptides were generated for the restimulation of the spleenocytes. For HlVgag a commercially available peptide mix of 130 peptides (JPT, Berlin) was used. The cytokines (IFN-gamma, IL-1beta, IL-2, IL-4, IL-5, IL-6, IL-10, IL-12, gCMSF and TNFalpha) were investigated by a multiplex assay kit from Invitrogen according to the manufacturer's protocol.

### Example 5

### Cellular Immune Response Based on EliSpot Data (Multiplex Assay)

At day 85 of the in vivo study (Example 2) mice were killed and the spleenocytes prepared for the analysis of 10 different cytokines. For the restimulation of the spleenocytes the complex [2 pg/mL] and re-CMV-VLPs [2 pg/mL] were used to receive initial data for a homologous and/or heterologous prime-boost regimen. The following proteins were verified for the complex: gpUL75, gpUL115, gpUL128, gpUL130 and gpUL131A. The cytokines (IFN-gamma, IL-1beta, IL-2, IL-4, IL-5, IL-6, IL-10, IL-12, gCMSF and TNFalpha) were investigated by a multiplex assay kit from Invitrogen according to the manufacturer's protocol. The restimulation with reCMV-VLPs of complex immunized mice led to an induction of a T-cell response verified by secretion of IL-4, gCMSF and IL-5. Cytokine secretion after restimulation with complex of the same sera was lower compared to the former one. Based on these results priming with complex (in the range of 1-10 ug/mouse) and boosting with reCMV-VLP (comprising further proteins such as gpUL83, gpUL55) could be a promising approach.

### Example 6

### Expression, Purification and Characterization of the Pentameric CMV Complex Comprising Proteins from Two Different Strains

According to the protein expression parameters defined based on systematic optimization (see Example 1 of PCT/EP2013/072717) the pentameric CMV complex comprising gpUL75 (gH-His)-gpUL115 (gL)-gpUL128-gpUL130-gpUL131A is produced in disposable shake flasks or wave bags (culture volume up to 25 L) in a variant of fall army worm Spodoptera frugiperda cells (Super-Sf9) by co-expression from a single baculovirus (SEQ ID NO: 67). This complex contains proteins from two different HCMV strains (Towne [NCBI FJ616285.1 and VR1814 [NCBI GU179289). The production parameters were as follows: Initial cell count at infection (CCI) of 2×10⁶ cells/ml, a multiplicity of infection (MOI) at 0.25 pfu/ml, incubation at 27° C. at 100 rpm. Harvest took place at day 3 post infection (55-65 h p.i.) with a viability around 80%. The production was controlled by daily sampling, determining cell count, average cell diameter, aggregation and viability. The complex containing supernatant was loaded on Ni²⁺-charged sepharose columns (GE Healthcare) in different scales dependent on the bulk volume. The complex was purified using a step gradient from zero to 500 mM imidazole over 23 column volumes (CV), equivalent to ˜460 ml. The different chromatographic fractions were analysed by biochemical methods. 150-300 μl of different fractions were precipitated with acetone, resuspended in 33 μl 20 mM Tris, 150 mM NaCl buffer, pH 7.4. For loading onto a 4-12% Bis-Tris NuPAGE gel (Invitrogen), 4× loading dye was added according to the manufacturer's protocol, followed by the electrophoreses for 15 min at 150 V and for 45 min at 180 V using MOPS running buffer. The gels were stained at least 1 h with SimplyBlue SafeStain reagent (Invitrogen) and destained with water. For concentration and further purification a 2^{nd} IMAC chromatography was performed using a further sepharose column (5 mL HisTrap) and a step gradient over 31 column volumes (CV) from 0-500 mM imidazole. The different fractions were analyzed like the 1^{st} IMAC step by Coomassie stained SDS-PAGE. All complex containing fractions were unified and concentrated on a PALL macrosep centrifugal device and dialysed against storage buffer containing 25 mM Tris, 150 mM NaCl, 3 mM KCl, pH 6.5, 3 mM EDTA. The purified, soluble complex was analyzed by SDS-PAGE (s. above) followed by Coomassie staining and densitometric analysis alongside of different amounts of BSA.

Exemplary expression vectors (pRBT136-x) for the generation of the pentameric CMV complex are illustrated in Table 1, above.

After expression and purification, the pentameric complex may be admixed with, e.g., a chelating agent and/or stabilizing agent as described herein.

### Example 7

### In Vivo Study in Mice Comprising Different Antigens

For the in vivo study Balb/C mice were used in a prime-boost-boost regimen. Each group contained 8 mice and each mouse received 5 pg, 10 pg or 20 pg protein per injection either with or without adjuvant. As negative control PBS was used, positive control was a inactivated AD169 lysate and for investigation of remaining baculoviruses (BV), the production virus with the according titer of remaining baculoviruses after purification was injected as well. Pre-immune sera were taken after 6 days quarantine of the mice (day 0). The first injection took place 10 days later followed by a booster injection at day 28. The first bleeding was done at day 36. The 2^{nd} booster injection was performed at day 48 followed by a further bleeding at day 55. The final bleeding took place at day 60 followed by the investigation of humoral and cellular immune response. Body weights were measured weekly (see FIG. 12).

### Example 8

### Humoral Immune Response Based on Neutralization Assay

The humoral immune response of the vaccine candidate based on the pentameric complex variants (SEQ ID: 18; SEQ ID:67 as well in combination with virus like particles [VLP; SEQ ID:6 on a carrier comprising UL86, UL85, UL48, UL83 and UL74)] was investigated by a neutralization assay of the mice sera from example 7 in comparison to sera from CMV negative and CMV positive human blood donors. A BAC (bacterial artificial chromosome)-reconstituted VR1814 strain carrying a GFP molecule for analytical reasons (fix-EGFP) as well as the TB40E strain were used for the infection of fibroblasts (MRC-5) and epithelial (ARPE-19) cells to visualize the neutralisation potential of the mouse sera from Example. 2×10⁴ cells/well were seeded into a 96 well plate in RPMI medium containing 10% FCS (fetal calf serum). A serum pool of 4 mice out of the 8 mice was generated and added in 2-fold serial dilutions (1:20 to 1:2560) in 100 μl/well RPMI/FCS medium. The above mentioned fix-EGFP VR1814 virus as well as TB40E were added in a tissue culture infectious dose (TCID) of 1000 virus molecules/well which was determined in a pre-assay. The 96 well plates were incubated for 8 days at 37° C. in a CO₂ controlled atmosphere. The determination of green cell was performed in a plate reader with the following parameters: fluorescein-filter [excitation 485/20, emission 530/25), bottom reading mode, time: 0.1 sec, 25× measurements/well after 96 h incubation. The neutralization potency was determined as the dilution of the sera able to show a 50% virus infection inhibition. The following controls were performed: cell control (cells+PBS), virus control (only infected cells) and 5 CMV positive and 5 CMV negative sera from human blood donors. Interestingly the neutralizing antibodies released from immunization with pentameric complex have as well the capability to reduce or inhibit virus entry via fibroblasts independent from the virus strain. These data are promising that a broad immune response based on vaccine containing the pentameric complex could be reached (see FIG. 8).

### Example 9

### Cellular Immune Response Based on EliSpot Data (Multiplex Assay)

At day 60 of the in vivo study (Example 2) mice were killed and the spleenocytes prepared for the analysis of 3 different cytokines. For the restimulation of the spleenocytes the AD169 virus lysate was used. The cytokines (IFN-gamma, IL-4, IL-5) were investigated by a multiplex assay kit from Invitrogen according to the manufacturer's protocol. Cytokine secretion after re-stimulation with a virus lysate containing a non-functional pentameric complex led to a Th-1 and Th-2 response. Re-stimulation with the functional protein could lead to promising cytokine induction. The adjuvant led to an increase in a specific Th-2 response measured via IL-4 secretion. Lower dosage (5 pg) of pentameric complex seems to be more beneficial than high dosage (10 pg). The baculovirus antigens led only to a negligible cytokine secretion (see FIG. 7).

### Example 10

### Improved Expression, Purification and Characterization of the Pentameric CMV Complex Comprising Proteins from Towne Strain

According to the protein expression parameters defined based on systematic optimization (see Example 1 of PCT/EP2013/072717) the pentameric CMV complex comprising gpUL75 (gH-His)-gpUL115 (gL)-gpUL128-gpUL130-gpUL131A is produced in disposable shake flasks or wave bags (culture volume up to 25 L) in a variant of fall army worm Spodoptera frugiperda cells (Super-Sf9) by co-expression from a single baculovirus (SEQ ID NO: 67). This complex contains proteins from HCMV strain Towne ([NCBI FJ616285.1). The production parameters were as follows: Initial cell count at infection (CCI) of 2×10⁶ cells/ml, a multiplicity of infection (MOI) in the range of 0.1 and 1, incubation at 27° C. at 100 rpm. Harvest took place at day 2-3 post infection (48-65 h p.i.) at a viability around 80% (see table 3). The production was controlled by daily sampling, determining cell count, average cell diameter, aggregation and viability. For the production in the wave bags different parameters were chosen: velocity between 19 and 22 rpm, angle of 6 and combined with a controlled oxygen supplies. The complex containing supernatant was loaded on Ni²⁺-charged sepharose columns (GE Healthcare) in different scales dependent on the bulk volume. The complex was purified using a step gradient from zero to 500 mM imidazole over 7 to 15 column volumes (CV). The different chromatographic fractions were analysed by biochemical methods. Either by direct loading of 15 μl or by an acetone precipitation of 150-300 μl of the different fractions (resuspended in 33 μl 20 mM Tris, 150 mM NaCl buffer, pH 7.4). For loading onto a 4-12% Bis-Tris NuPAGE gel (Invitrogen), 4× loading dye was added according to the manufacturer's protocol, followed by the electrophoreses for 15 min at 150 V and for 45 min at 180 V using MOPS running buffer. The gels were stained at least 1 h with SimplyBlue SafeStain reagent (Invitrogen) and destained with water. To reduce the amount of remaining baculovirus an anion-exchange chromatography in negative mode was performed. The flowthrough containing the pentameric complex was further concentrated and purified by a 2^{nd} affinity chromatography using a further sepharose column (scale depends on the volume) and a step gradient over 15-30 column volumes (CV) from 0-500 mM imidazole. The different fractions were analyzed like the 1^{st} IMAC step by Coomassie stained SDS-PAGE. All complex containing fractions were unified and quality controlled. A concentration with PALL macrosep centrifugal devices could be integrated. Displacement of imidazole were performed by several dialysis steps. The first dialysis buffer contains 20 mM EDTA which is then reduced to a lower amount 0-3 mM EDTA. A complete buffer switch such into PBS is also possible. For storage and stabilization of the complex different chemical agents such as Tween20, Tween80, glycerol could be added. The purified, soluble complex was analyzed by SDS-PAGE (s. above) followed by Coomassie staining and densitometric analysis alongside of different amounts of BSA. Protein concentration was determined by BCA assay as well as the remaining baculoviruses. Remaining baculoviral genomes are determined by qPCR based on a viral reference gene (IE-1), as well as by fluorescence measurement of viral genomes (and nucleic acids in general) and proteins in viral capsids with the virus counter (Virocyt) using a combination of equilibrium dyes. This “combination” approach by determination of simultaneous absorbance of nucleic acids and viral membrane proteins allows the detection of total amount of viral particles whereas the infectious particles were measured by plaque assay. An Endosafe device from Charles River (PTS20F) based on the LAL assay was used for endotoxin determination using validated single-use cartridges. Quant-iT™ PicoGreen® Assay from Invitrogen (P7589) was used for determination of dsDNA content in the end product.

Product identity, at the end of the DSP process (IMAC-AEX-IMAC), was confirmed via a direct ELISA. The complex was verified with an α-gH-antibody (Santa Cruz, sc-58113) and an α-His-antibody (AbD Serotec, MCA1396) and detected with an α-mouse-HRP antibody (Cell Signaling, 7076S) (see FIGS. 9 and 10).

After expression and purification, the pentameric complex may be admixed with, e.g., a chelating agent and/or stabilizing agent as described herein (see FIG. 11).

1. A pentameric complex composed of CMV proteins UL128, UL130, UL131A, gH (UL75) and gL (UL115) obtainable by the method, comprising
(i) co-expressing CMV proteins UL128, UL130, UL131A, gH (UL75) and gL (UL115) in a host cell by using baculovirus;
(ii) purifying the pentameric complex from host cells and/or supernatant obtained from said co-expression; and
(iii) storing the purified pentameric complex in a buffer solution comprising a chelating agent and/or a stabilizing agent.

2. The pentameric complex according to claim 1, wherein the host cell is an insect cell or mammalian cell.

3. The pentameric complex according to claim 2, wherein the insect cell is Sf9, Sf21, HighFive, S2, Super Sf9-1, Super Sf9-2, or Super Sf-9-3.

4. The pentameric complex according to claim 1, wherein the co-expression step comprises
(i) infecting host cells with a baculovirus expressing said proteins and having a titer of about 10⁷ pfu/mL or higher when infecting said host cell having a cell count at infection of about 2*10⁶ cells/mL;
(ii) cultivating said host cells under suitable conditions, and
(iii) harvesting said host cells and/or supernatant between 56-65 h post infection.

5. The pentameric complex according to claim 1, wherein said host cells are infected between day 15 and day 50 after thawing and culturing.

6. The pentameric complex according to claim 1, wherein purification comprises ion exchange chromatography, hydrophobic interaction chromatography, size exclusion chromatography and/or affinity chromatography.

7. The pentameric complex according to claim 1, wherein the chelating agent is Ethylenediaminetetraacetic acid (EDTA) or ethylene glycol tetraacetic acid (EGTA).

8. The pentameric complex according to claim 7, wherein EDTA is present in said buffer solution at a concentration of 20 mM or less, such as 3 mM or less.

9. The pentameric complex according to claim 1, wherein the stabilizing agent is polyethylene glycol, arginine, sorbitol, glycerol, sucrose and/or NP-40.

10. The pentameric complex according to claim 1, wherein said buffer solution comprises Tris buffer, NaCl, KCl.

11. The pentameric complex according to claim 1, wherein open reading frames (ORFs) encoding CMV proteins UL128, UL130, UL131A, gH and gL are on a single vector.

12. The pentameric complex according to claim 11, wherein the vector contains elements for propagation in bacteria (E. coli), yeast (S. cerevisiae), insect cells and/or mammalian cells.

13. The pentameric complex according to claim 11, wherein the ORFs are located in the following order from 5′ to 3′ in said vector:
(i) gH, gL, UL128, UL130, UL131A; or
(ii) gL, UL128, UL130, UL131A, gH.

14. The pentameric complex according to claim 13, wherein
(a) in (i) the gH ORF is transcribed in 3′ direction, the gL ORF is transcribed in 5′ direction, the UL128 ORF is transcribed in 3′ direction, the UL130 ORF is transcribed in 3′ direction, and the UL131A ORF is transcribed in 3′ direction;
(b) in (i) gH ORF is transcribed in 3′ direction, the gL ORF is transcribed in 3′ direction, the UL128 ORF is transcribed in 3′ direction, the UL130 ORF is transcribed in 3′ direction, and the UL131A ORF is transcribed in 3′ direction;
(c) in (ii) gL ORF is transcribed in 5′ direction, the UL128 ORF is transcribed in 3′ direction, the UL130 ORF is transcribed in 3′ direction, the UL131A ORF is transcribed in 3′ direction, and the gH ORF is transcribed in 3′ direction

15. The pentameric complex according to claim 11, wherein each of said ORFs is driven by the p10 promoter, polh promoter, IE-1 promoter, mCMV promoter, vp39 promoter, lef2 promoter, CAG promoter, or HepB SV40 promoter and followed by a terminator sequence such as HSVtk terminator or SV40 terminator

16. The pentameric complex according to claim 1, wherein at least one of said proteins comprises a tag.

17. The pentameric complex according to claim 16, wherein said tag is a His-Tag, Strep-Tag, a His-Strep-tag, StrepII-Tag, Softag 1, TC-tag, myc-Tag, FLAG-tag, HA-tag, V5-tag, Avi-tag, Calmodulin-tag, polyglutamate-tag, amyloid beta-tag, GST-tag, MBP-tag or S-tag.

18. The pentameric complex according to claim 1, wherein one or more of said proteins comprises PRESCISSION protease or PRESCISSION and TEV protease.

19. The pentameric complex according to claim 1, wherein in said baculovirus v-cath and/or ChiA activity is functionally disrupted.

20. A composition comprising the pentameric complex according to claim 1.

21. The pentameric complex according to claim 1, wherein the complex is capable of inducing neutralization activity that inhibits both epithelial/endothelial (Epi/EC) and fibroblast infection.

22. A method for the production of a pentameric complex composed of CMV proteins UL128, UL130, UL131A, gH (UL75) and gL (UL115), comprising
(i) co-expressing CMV proteins UL128, UL130, UL131A, gH (UL75) and gL (UL115) in a host cell by using baculovirus;
(ii) purifying the pentameric complex from host cells and/or supernatant obtained from said co-expression; and
(iii) storing the purified pentameric complex in a buffer solution comprising a chelating agent and/or a stabilizing agent.

23. The pentameric complex according to claim 1, wherein one or more of said proteins comprises additional B- and/or T-cell epitopes

24. The pentameric complex according to claim 23, wherein said T-cell epitope is a CD4 T-cell epitope or a CD8 T-cell epitope.

25. The pentameric complex according to claim 23, wherein said epitope is any one of the epitopes shown in SEQ ID NOs: 22-66.

26. An immunogenic composition comprising the pentameric complex according to claim 1 and a pharmaceutically acceptable carrier or adjuvant.

27. The pentameric complex according to claim 1, wherein at least one, two, three or four of said proteins is from a CMV strain other than the CMV strain from which the remaining proteins are from.

28. The pentameric complex according to claim 27, wherein the CMV proteins are from CMV strain Towne, Towne having the genome as deposited with NCBI GenBank under accession number FJ616285.1, Toledo (GU937742.1), AD169 (FJ527563), Merlin (AY446894.2), TB40/E (KF297339.1), VR1814 (GU179289).

29. The immunogenic composition of claim 26, further comprising gB protein, gM protein, pp65 protein, IE-1 protein, dimer of gL/gH protein, dimer of gM/gN protein, trimer of gL/gH/gO, virus-like particles (VLPs) comprising one or more capsid or capsid precursor proteins, one or more surface proteins from CMV, and/or or one or more tegument proteins.

30. The immunogenic composition of claim 26, further comprising a nucleic acid molecule encoding gB, gM, pp65, IE-1 or IE-2

31. An immunogenic composition comprising a vector encoding the pentameric complex according to claim 1.

32. The immunogenic composition according to claim 31, wherein said vector is DNA- or RNA-based.

33. The immunogenic composition according to claim 26, further comprising a modified vaccinia virus Ankara (MVA) comprising one or more proteins of the CMV pentameric complex composed of UL128, UL130, UL131, gH (UL75) and gL (UL115).

34. The immunogenic composition according to claim 26 for use in a method of vaccinating a subject against CMV, comprising administering as priming composition said immunogenic composition and as boosting composition a (i) gH/gL dimer, (ii) a UL130/UL131A-dimer, (iii) gM/gN dimer (iv) a gH/gL/UL128/UL130/UL131A-pentamer, (v) gB, (vi) gM, (vii) pp65, (viii) IE-1, (ix) IE-2, (x) a modified vaccinia virus Ankara (MVA) comprising one or more proteins of the CMV pentameric complex composed of UL128, UL130, UL131, gH (UL75) and gL (UL115), (xi) a modified vaccinia virus Ankara (MVA) comprising gB, a gH/gL dimer, pp65 protein or IE-1 protein, (xii) virus-like particles (VLPs) comprising one or more capsid or capsid precursor proteins, one or more surface proteins from CMV, or one or more tegument proteins, (xiii) nucleic acid sequence encoding any one of the compounds as defined in (i) to (xii), (xiv) peptides from flagellin, (xv) CpG motifs, and/or (xvi) LCMV to said subject.

35. The immunogenic composition according to claim 34, wherein said boosting composition is used as priming composition and said priming composition is used as boosting composition.

36. A vector comprising open reading frames (ORFs) encoding CMV proteins UL128, UL130, UL131, gH and gL.

37. The vector according to claim 36, wherein the vector contains elements for propagation in bacteria (e.g. E. coli), yeast (e.g. S. cerevisiae), insect cells and/or mammalian cells.

38. The vector according to claim 36, wherein said vector is a Baculovirus vector or a Baculovirus BacMam vector.

39. The vector according to claim 36, wherein in said baculovirus vector the v-cath and/or ChiA gene is functionally disrupted.

40. The vector according to claim 36, wherein the ORFs are located in the following order from 5′ to 3′ in said vector:
(a) gH, gL, UL128, UL130, UL131; or.
(b) gL, UL128, UL130, UL131, gH.

41. The vector according to claim 36, wherein
(a) in (i) the gH ORF is transcribed in 3′ direction, the gL ORF is transcribed in 5′ direction, the UL128 ORF is transcribed in 3′ direction, the UL130 ORF is transcribed in 3′ direction, and the UL131 ORF is transcribed in 3′ direction;
(b) in (i) gH ORF is transcribed in 3′ direction, the gL ORF is transcribed in 3′ direction, the UL128 ORF is transcribed in 3′ direction, the UL130 ORF is transcribed in 3′ direction, and the UL131 ORF is transcribed in 3′ direction;
(c) in (ii) gL ORF is transcribed in 5′ direction, the UL128 ORF is transcribed in 3′ direction, the UL130 ORF is transcribed in 3′ direction, the UL131 ORF is transcribed in 3′ direction, and the gH ORF is transcribed in 3′ direction.

42. The vector according to claim 36, wherein each of said ORFs is driven by p10 promoter, polh promoter, IE-1 promoter, mCMV promoter, vp39 promoter, lef2 promoter, CAG promoter, or HepB SV40 promoter and followed by a terminator sequence such as HSVtk terminator or SV40 terminator.

43. An immunogenic comprising the vector according to claim 36 and a pharmaceutically acceptable carrier or adjuvant.


SEQUENCE LISTING (72 sequences)

SEQ ID NO: 1 - protein, 716 aa, Cytomegalovirus
MRPGLPSYLIVLAVCLLSHLLSSRYGAEAISEPLDKAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGEQPTTVPPPIDLSIPHVWMPPQTTPHGWTESHTTSGLHRPHFNQTCILFDGHDLLFSTVTPCLHQGFYLIDELRYVKITLTEDFFVVTVSIDDDTPMLLIFGHLPRVLFKAPYQRDNFILRQTEKHELLVLVKKDQLNRHSYLKDPDFLDAALDFNYLDLSALLRNSFHRYAVDVLKSGRCQMLDRRTVEMAFAYALALFAAARQEEAGAQVSVPRALDRQAALLQIQEFMITCLSQTPPRTTLLLYPTAVDLAKRALWTPNQITDITSLVRLVYILSKQNQQHLIPQWALRQIADFALKLHKTHLASFLSAFARQELYLMGSLVHSMLVHTTERREIFIVETGLCSLAELSHFTQLLAHPHHEYLSDLYTPCSSSGRRDHSLERLTRLFPDATVPTTVPAALSILSTMQPSTLETFPDLFCLPLGESFSALTVSEHVSYVVTNQYLIKGISYPVSTTVVGQSLIITQTDSQTKCELTRNMHTTHSITAALNISLENCAFCQSALLEYDDTQGVINIMYMHDSDDVLFALDPYNEVVVSSPRTHYLMLLKNGTVLEVTDVVVDATDS

SEQ ID NO: 2 - protein, 278 aa, Cytomegalovirus
MCRRPDCGFSFSPGPVALLWCCLLLPIVSSATVSVAPTVAEKVPAECPELTRRCLLGEVFQGDKYESWLRPLVNVTRRDGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 3 - protein, 171 aa, Cytomegalovirus
MSPKNLTPFLTALWLLLGHSRVPRVRAEECCEFINVNHPPERCYDFKMCNRFTVALRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQ

SEQ ID NO: 4 - protein, 213 aa, Cytomegalovirus
MLRLLLRHHFHCLLLCAVWATPCLASPWSTLTANQNPSPPWSKLTYSKPHDAATFYCPFLYPSPPRSPLQFSGFQRVLTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLI

SEQ ID NO: 5 - protein, 128 aa, Cytomegalovirus
MRLCRVWLSVCLCAVVLGQCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFA

SEQ ID NO: 6 - DNA, 11317 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggtttgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggttttactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgaccgtctgctgatgatgtccgtgtacgctctgtccgctatcatcggcatctacctgctgtaccgtatgctgaagacctgctaaggcgccaagctttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggggcctctaaacgggtcttgaggggttttttgctgaaaggaggaactatatccggatctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagctgacattcatccggggtcagcaccgtttctgcggactggctttctacgtgttccgcttcctttagcagcccttgcgccctgagtgcttgcggcagcgtgaagctaattctgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgtcccccaagaacctgacccccttcctgaccgctctgtggctgctgctgggccactcccgtgtgcctcgtgtgcgtgctgaggaatgctgcgagttcatcaacgtgaaccacccccccgagcgctgctacgacttcaagatgtgcaaccgtttcaccgtggctctgcgttgccccgacggcgaagtgtgctactcccccgaaaagaccgctgagatccgtggtatcgtgaccaccatgacccactccctgacccgtcaggtggtgcacaacaagctgacctcttgcaactacaaccccctgtacctcgaggctgacggtcgtatccgttgcggcaaggtcaacgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccttggtccaccctgaccgctaaccagaacccctccccaccctggtccaagctgacctactccaagccccacgacgctgctaccttctactgccccttcctgtacccatccccccctcgttccccactgcagttctccggtttccagcgtgtgctgaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctcgagtcctgggctcacgtgttccgtgactactccgtgtccttccaagtgcgcctgaccttcaccgaggctaacaaccagacctacaccttctgcacccaccccaacctgatcgtgtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtctgtgccgtgtgtggctgtccgtgtgcctgtgcgctgtggtgctgggccagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccctcgagttctccgtgcgtctgttcgctaactaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 7 - DNA, 11317 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggtttgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggttttactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgaccgtctgctgatgatgtccgtgtacgctctgtccgctatcatcggcatctacctgctgtaccgtatgctgaagacctgctaaggcgccaagctttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggggcctctaaacgggtcttgaggggttttttgctgaaaggaggaactatatccggatctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagctgacattcatccggggtcagcaccgtttctgcggactggctttctacgtgttccgcttcctttagcagcccttgcgccctgagtgcttgcggcagcgtgaagctaattctgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgaactccaaggacctgacccccttcctgaccaccctgtggctgctgctggaccactcccgtgtgcctcgtgtgcgtgctgaggaatgctgcgaagtgatcaacgtgaaccacccccccgagcgctgctacgacttcaagatgtgcaacctgttcaccgtggctctgcgttgccccgacggcgaagtgtgctactcccccgaaaagaccgctgagatccgtggtatcgtgaccaccatgacccactccctgacccgtcaagtgatccacaacaagctgaccagctgcaactacaaccccctgtacctcgaggctgacggtcgtatccgttgcggcaaggtgtccgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccttggtccaccctgaccgctaaccagaacccctccccaccctggtccaagctgacctactccaagccccacgacgctgctaccttctactgccccttcctgtacccatccccccctcgttccccactgcagttctccggtttccagcgtgtgctgaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctcgagtcctgggctcacgtgttccgtgactactccgtgtccttccaagtgcgcctgaccttcaccgaggctaacaaccagacctacaccttctgcacccaccccaacctgatcgtgtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtctgtgccgtgtgtggctgtccgtgtgcctgtgcgctgtggtgctgggccagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccctcgagttctccgtgcgtctgttcgctaactaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 8 - DNA, 11363 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggtttgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggttttactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgaccgtctgctgatgatgtccgtgtacgctctgtccgctatcatcggcatctacctgctgtaccgtatgctgaagacctgctaaggcgccaagctttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggggcctctaaacgggtcttgaggggttttttgctgaaaggaggaactatatccggatctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagctgacattcatccggggtcagcaccgtttctgcggactggctttctacgtgttccgcttcctttagcagcccttgcgccctgagtgcttgcggcagcgtgaagctaattctgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctccggtaccgcatgctatgcttagtgatggtggtggtgatggtggtggctgcctccccccccagacccccctccgccgcttccgcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgtcccccaagaacctgacccccttcctgaccgctctgtggctgctgctgggccactcccgtgtgcctcgtgtgcgtgctgaggaatgctgcgagttcatcaacgtgaaccacccccccgagcgctgctacgacttcaagatgtgcaaccgtttcaccgtggctctgcgttgccccgacggcgaagtgtgctactcccccgaaaagaccgctgagatccgtggtatcgtgaccaccatgacccactccctgacccgtcaggtggtgcacaacaagctgacctcttgcaactacaaccccctgtacctcgaggctgacggtcgtatccgttgcggcaaggtcaacgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccttggtccaccctgaccgctaaccagaacccctccccaccctggtccaagctgacctactccaagccccacgacgctgctaccttctactgccccttcctgtacccatccccccctcgttccccactgcagttctccggtttccagcgtgtgctgaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctcgagtcctgggctcacgtgttccgtgactactccgtgtccttccaagtgcgcctgaccttcaccgaggctaacaaccagacctacaccttctgcacccaccccaacctgatcgtgtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtctgtgccgtgtgtggctgtccgtgtgcctgtgcgctgtggtgctgggccagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccctcgagttctccgtgcgtctgttcgctaactaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 9 - DNA, 11363 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggtttgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggttttactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgaccgtctgctgatgatgtccgtgtacgctctgtccgctatcatcggcatctacctgctgtaccgtatgctgaagacctgctaaggcgccaagctttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggggcctctaaacgggtcttgaggggttttttgctgaaaggaggaactatatccggatctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagctgacattcatccggggtcagcaccgtttctgcggactggctttctacgtgttccgcttcctttagcagcccttgcgccctgagtgcttgcggcagcgtgaagctaattctgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctccggtaccgcatgctatgcttagtgatggtggtggtgatggtggtggctgcctccccccccagacccccctccgccgcttccgcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgaactccaaggacctgacccccttcctgaccaccctgtggctgctgctggaccactcccgtgtgcctcgtgtgcgtgctgaggaatgctgcgaagtgatcaacgtgaaccacccccccgagcgctgctacgacttcaagatgtgcaacctgttcaccgtggctctgcgttgccccgacggcgaagtgtgctactcccccgaaaagaccgctgagatccgtggtatcgtgaccaccatgacccactccctgacccgtcaagtgatccacaacaagctgaccagctgcaactacaaccccctgtacctcgaggctgacggtcgtatccgttgcggcaaggtgtccgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccttggtccaccctgaccgctaaccagaacccctccccaccctggtccaagctgacctactccaagccccacgacgctgctaccttctactgccccttcctgtacccatccccccctcgttccccactgcagttctccggtttccagcgtgtgctgaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctcgagtcctgggctcacgtgttccgtgactactccgtgtccttccaagtgcgcctgaccttcaccgaggctaacaaccagacctacaccttctgcacccaccccaacctgatcgtgtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtctgtgccgtgtgtggctgtccgtgtgcctgtgcgctgtggtgctgggccagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccctcgagttctccgtgcgtctgttcgctaactaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 10 - DNA, 11432 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggtttgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggttttactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgaccgtctgctgatgatgtccgtgtacgctctgtccgctatcatcggcatctacctgctgtaccgtatgctgaagacctgctaaggcgccaagctttgtcgagaagtactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggggcctctaaacgggtcttgaggggttttttgctgaaaggaggaactatatccggatctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagctgacattcatccggggtcagcaccgtttctgcggactggctttctacgtgttccgcttcctttagcagcccttgcgccctgagtgcttgcggcagcgtgaagctaattctgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcttagtgatggtggtggtgatggtggtggctgcctccccccccagacccccctccgccgcttcctttttcgaactgcgggtggctccagctacctccgccgggcccctggaacagaacttccagggcgccttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgtcccccaagaacctgacccccttcctgaccgctctgtggctgctgctgggccactcccgtgtgcctcgtgtgcgtgctgaggaatgctgcgagttcatcaacgtgaaccacccccccgagcgctgctacgacttcaagatgtgcaaccgtttcaccgtggctctgcgttgccccgacggcgaagtgtgctactcccccgaaaagaccgctgagatccgtggtatcgtgaccaccatgacccactccctgacccgtcaggtggtgcacaacaagctgacctcttgcaactacaaccccctgtacctcgaggctgacggtcgtatccgttgcggcaaggtcaacgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccttggtccaccctgaccgctaaccagaacccctccccaccctggtccaagctgacctactccaagccccacgacgctgctaccttctactgccccttcctgtacccatccccccctcgttccccactgcagttctccggtttccagcgtgtgctgaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctcgagtcctgggctcacgtgttccgtgactactccgtgtccttccaagtgcgcctgaccttcaccgaggctaacaaccagacctacaccttctgcacccaccccaacctgatcgtgtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtctgtgccgtgtgtggctgtccgtgtgcctgtgcgctgtggtgctgggccagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccctcgagttctccgtgcgtctgttcgctaactaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 11 - DNA, 11432 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggtttgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggttttactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgaccgtctgctgatgatgtccgtgtacgctctgtccgctatcatcggcatctacctgctgtaccgtatgctgaagacctgctaaggcgccaagctttgtcgagaagtactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggggcctctaaacgggtcttgaggggttttttgctgaaaggaggaactatatccggatctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagctgacattcatccggggtcagcaccgtttctgcggactggctttctacgtgttccgcttcctttagcagcccttgcgccctgagtgcttgcggcagcgtgaagctaattctgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcttagtgatggtggtggtgatggtggtggctgcctccccccccagacccccctccgccgcttcctttttcgaactgcgggtggctccagctacctccgccgggcccctggaacagaacttccagggcgccttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgaactccaaggacctgacccccttcctgaccaccctgtggctgctgctggaccactcccgtgtgcctcgtgtgcgtgctgaggaatgctgcgaagtgatcaacgtgaaccacccccccgagcgctgctacgacttcaagatgtgcaacctgttcaccgtggctctgcgttgccccgacggcgaagtgtgctactcccccgaaaagaccgctgagatccgtggtatcgtgaccaccatgacccactccctgacccgtcaagtgatccacaacaagctgaccagctgcaactacaaccccctgtacctcgaggctgacggtcgtatccgttgcggcaaggtgtccgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccttggtccaccctgaccgctaaccagaacccctccccaccctggtccaagctgacctactccaagccccacgacgctgctaccttctactgccccttcctgtacccatccccccctcgttccccactgcagttctccggtttccagcgtgtgctgaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctcgagtcctgggctcacgtgttccgtgactactccgtgtccttccaagtgcgcctgaccttcaccgaggctaacaaccagacctacaccttctgcacccaccccaacctgatcgtgtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtctgtgccgtgtgtggctgtccgtgtgcctgtgcgctgtggtgctgggccagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccctcgagttctccgtgcgtctgttcgctaactaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 12 - DNA, 11338 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggtttgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggttttactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgaccgtctgctgatgatgtccgtgtacgctctgtccgctatcatcggcatctacctgctgtaccgtatgctgaagacctgctaaggcgccaagctttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggggcctctaaacgggtcttgaggggttttttgctgaaaggaggaactatatccggatctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagctgacattcatccggggtcagcaccgtttctgcggactggctttctacgtgttccgcttcctttagcagcccttgcgccctgagtgcttgcggcagcgtgaagctaattctgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgtcccccaagaacctgacccccttcctgaccgctctgtggctgctgctgggccactcccgtgtgcctcgtgtgcgtgctgaggaatgctgcgagttcatcaacgtgaaccacccccccgagcgctgctacgacttcaagatgtgcaaccgtttcaccgtggctctgcgttgccccgacggcgaagtgtgctactcccccgaaaagaccgctgagatccgtggtatcgtgaccaccatgacccactccctgacccgtcaggtggtgcacaacaagctgacctcttgcaactacaaccccctgtacctcgaggctgacggtcgtatccgttgcggcaaggtcaacgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccctggttcaccctgaccgctaaccagaacccctccccaccctggtccaagctgacctaccccaagccccacgacgctgctaccttctactgccccttcctgtacccatccccacctcgttccccatcccagttctccggtttccagcgtgtgtccaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctggaatcctgggctcacgtgttccgtgactactccgtgtccttccaagtgcgcctgaccttcaccgaggctaacaaccagacctacaccttctgcacccaccccaacctgatcgtgtaagtcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccggatccgccaccatgcgtctgtgccgtgtgtggctgtccgtgtgcctgtgcgctgtggtgctgggacagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccttggagttctccgtgcgtctgttcgccaactaataaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 13 - DNA, 11338 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggtttgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggttttactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgaccgtctgctgatgatgtccgtgtacgctctgtccgctatcatcggcatctacctgctgtaccgtatgctgaagacctgctaaggcgccaagctttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggggcctctaaacgggtcttgaggggttttttgctgaaaggaggaactatatccggatctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagctgacattcatccggggtcagcaccgtttctgcggactggctttctacgtgttccgcttcctttagcagcccttgcgccctgagtgcttgcggcagcgtgaagctaattctgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgaactccaaggacctgacccccttcctgaccaccctgtggctgctgctggaccactcccgtgtgcctcgtgtgcgtgctgaggaatgctgcgaagtgatcaacgtgaaccacccccccgagcgctgctacgacttcaagatgtgcaacctgttcaccgtggctctgcgttgccccgacggcgaagtgtgctactcccccgaaaagaccgctgagatccgtggtatcgtgaccaccatgacccactccctgacccgtcaagtgatccacaacaagctgaccagctgcaactacaaccccctgtacctcgaggctgacggtcgtatccgttgcggcaaggtgtccgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccctggttcaccctgaccgctaaccagaacccctccccaccctggtccaagctgacctaccccaagccccacgacgctgctaccttctactgccccttcctgtacccatccccacctcgttccccatcccagttctccggtttccagcgtgtgtccaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctggaatcctgggctcacgtgttccgtgactactccgtgtccttccaagtgcgcctgaccttcaccgaggctaacaaccagacctacaccttctgcacccaccccaacctgatcgtgtaagtcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccggatccgccaccatgcgtctgtgccgtgtgtggctgtccgtgtgcctgtgcgctgtggtgctgggacagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccttggagttctccgtgcgtctgttcgccaactaataaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 14 - DNA, 11503 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggtttgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggttttactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgaccgtctgctgatgatgtccgtgtacgctctgtccgctatcatcggcatctacctgctgtaccgtatgctgaagacctgcggttcctccggtggcgccggtggctccggttcctctggcggttctggttcctccgctcaacttgagaaagaactgcaggctctcgaaaaagaaaacgcccaactcgaatgggagctgcaagccttggaaaaagaactggcccaattggaaaaagaaaaccaggctttggaatgggagctggctcagtaaggcgccaagctttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggggcctctaaacgggtcttgaggggttttttgctgaaaggaggaactatatccggatctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagctgacattcatccggggtcagcaccgtttctgcggactggctttctacgtgttccgcttcctttagcagcccttgcgccctgagtgcttgcggcagcgtgaagctaattctgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgtcccccaagaacctgacccccttcctgaccgctctgtggctgctgctgggccactcccgtgtgcctcgtgtgcgtgctgaggaatgctgcgagttcatcaacgtgaaccacccccccgagcgctgctacgacttcaagatgtgcaaccgtttcaccgtggctctgcgttgccccgacggcgaagtgtgctactcccccgaaaagaccgctgagatccgtggtatcgtgaccaccatgacccactccctgacccgtcaggtggtgcacaacaagctgacctcttgcaactacaaccccctgtacctcgaggctgacggtcgtatccgttgcggcaaggtcaacgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccttggtccaccctgaccgctaaccagaacccctccccaccctggtccaagctgacctactccaagccccacgacgctgctaccttctactgccccttcctgtacccatccccccctcgttccccactgcagttctccggtttccagcgtgtgctgaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctcgagtcctgggctcacgtgttccgtgactactccgtgtccttccaagtgcgcctgaccttcaccgaggctaacaaccagacctacaccttctgcacccaccccaacctgatcgtgtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtctgtgccgtgtgtggctgtccgtgtgcctgtgcgctgtggtgctgggccagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccctcgagttctccgtgcgtctgttcgctaactaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 15 - DNA, 11503 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggtttgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggttttactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgaccgtctgctgatgatgtccgtgtacgctctgtccgctatcatcggcatctacctgctgtaccgtatgctgaagacctgcggttcctccggtggcgccggtggctccggttcctctggcggttctggttcctccgctcaacttgagaaagaactgcaggctctcgaaaaagaaaacgcccaactcgaatgggagctgcaagccttggaaaaagaactggcccaattggaaaaagaaaaccaggctttggaatgggagctggctcagtaaggcgccaagctttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggggcctctaaacgggtcttgaggggttttttgctgaaaggaggaactatatccggatctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagctgacattcatccggggtcagcaccgtttctgcggactggctttctacgtgttccgcttcctttagcagcccttgcgccctgagtgcttgcggcagcgtgaagctaattctgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgaactccaaggacctgacccccttcctgaccaccctgtggctgctgctggaccactcccgtgtgcctcgtgtgcgtgctgaggaatgctgcgaagtgatcaacgtgaaccacccccccgagcgctgctacgacttcaagatgtgcaacctgttcaccgtggctctgcgttgccccgacggcgaagtgtgctactcccccgaaaagaccgctgagatccgtggtatcgtgaccaccatgacccactccctgacccgtcaagtgatccacaacaagctgaccagctgcaactacaaccccctgtacctcgaggctgacggtcgtatccgttgcggcaaggtgtccgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccttggtccaccctgaccgctaaccagaacccctccccaccctggtccaagctgacctactccaagccccacgacgctgctaccttctactgccccttcctgtacccatccccccctcgttccccactgcagttctccggtttccagcgtgtgctgaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctcgagtcctgggctcacgtgttccgtgactactccgtgtccttccaagtgcgcctgaccttcaccgaggctaacaaccagacctacaccttctgcacccaccccaacctgatcgtgtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtctgtgccgtgtgtggctgtccgtgtgcctgtgcgctgtggtgctgggccagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccctcgagttctccgtgcgtctgttcgctaactaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 16 - DNA, 11619 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggtttgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggttttactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgaccgtctgctgatgatgtccgtgtacgctctgtccgctatcatcggcatctacctgctgtaccgtatgctgaagacctgcggttcctccggtggcgccggtggctccggttcctctggcggttctggttcctccgctcaacttgagaaagaactgcaggctctcgaaaaagaaaacgcccaactcgaatgggagctgcaagccttggaaaaagaactggcccaattggaaaaagaaaaccaggctttggaatgggagctggctcagtaaggcgccaagctttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggggcctctaaacgggtcttgaggggttttttgctgaaaggaggaactatatccggatctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagctgacattcatccggggtcagcaccgtttctgcggactggctttctacgtgttccgcttcctttagcagcccttgcgccctgagtgcttgcggcagcgtgaagctaattctgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcttagtgatggtggtggtgatggtggtggctgcctccccccccagacccccctccgccgcttcctttttcgaactgcgggtggctccagctacctccgccgggcccctggaacagaacttccagggcgccttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgtcccccaagaacctgacccccttcctgaccgctctgtggctgctgctgggccactcccgtgtgcctcgtgtgcgtgctgaggaatgctgcgagttcatcaacgtgaaccacccccccgagcgctgctacgacttcaagatgtgcaaccgtttcaccgtggctctgcgttgccccgacggcgaagtgtgctactcccccgaaaagaccgctgagatccgtggtatcgtgaccaccatgacccactccctgacccgtcaggtggtgcacaacaagctgacctcttgcaactacaaccccctgtacctcgaggctgacggtcgtatccgttgcggcaaggtcaacgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccttggtccaccctgaccgctaaccagaacccctccccaccctggtccaagctgacctactccaagccccacgacgctgctaccttctactgccccttcctgtacccatccccccctcgttccccactgcagttctccggtttccagcgtgtgctgaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctcgagtcctgggctcacgtgttccgtgactactccgtgtccttccaagtgcgcctgaccttcaccgaggctaacaaccagacctacaccttctgcacccaccccaacctgatcgtgtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtctgtgccgtgtgtggctgtccgtgtgcctgtgcgctgtggtgctgggccagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccctcgagttctccgtgcgtctgttcgctaactaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 17 - DNA, 11619 bp, artificial; identical to SEQ ID NO: 16
  synthetic sequence for expression of pentameric CMV complex

SEQ ID NO: 18 - DNA, 11787 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggtttgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgactccggcgccggaagcggcggaggggggtctggggggggaggcagccaccaccatcaccaccaccatcactaagaattcaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggggcctctaaacgggtcttgaggggttttttgctgaaaggaggaactatatccggatctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagctgacattcatccggggtcagcaccgtttctgcggactggctttctacgtgttccgcttcctttagcagcccttgcgccctgagtgcttgcggcagcgtgaagctaattctgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgtcccccaagaacctgacccccttcctgaccgctctgtggctgctgctgggccactcccgtgtgcctcgtgtgcgtgctgaggaatgctgcgagttcatcaacgtgaaccacccccccgagcgctgctacgacttcaagatgtgcaaccgtttcaccgtggctctgcgttgccccgacggcgaagtgtgctactcccccgaaaagaccgctgagatccgtggtatcgtgaccaccatgacccactccctgacccgtcaggtggtgcacaacaagctgacctcttgcaactacaaccccctgtacctcgaggctgacggtcgtatccgttgcggcaaggtcaacgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccttggtccaccctgaccgctaaccagaacccctccccaccctggtccaagctgacctactccaagccccacgacgctgctaccttctactgccccttcctgtacccatccccccctcgttccccactgcagttctccggtttccagcgtgtgctgaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctcgagtcctgggctcacgtgttccgtgactactccgtgtccttccaagtgcgcctgaccttcaccgaggctaacaaccagacctacaccttctgcacccaccccaacctgatcgtgtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtctgtgccgtgtgtggctgtccgtgtgcctgtgcgctgtggtgctgggccagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccctcgagttctccgtgcgtctgttcgctaactaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 19 - DNA, 11848 bp, artificial
  synthetic sequence for expression of pentameric CMV complex
gttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggtttgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgactccggcgccctggaagttctgttccaggggcccggcggaggtagctggagccacccgcagttcgaaaaaggaagcggcggaggggggtctggggggggaggcagccaccaccatcaccaccaccatcactaagaattcaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggggcctctaaacgggtcttgaggggttttttgctgaaaggaggaactatatccggatctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagctgacattcatccggggtcagcaccgtttctgcggactggctttctacgtgttccgcttcctttagcagcccttgcgccctgagtgcttgcggcagcgtgaagctaattctgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgtcccccaagaacctgacccccttcctgaccgctctgtggctgctgctgggccactcccgtgtgcctcgtgtgcgtgctgaggaatgctgcgagttcatcaacgtgaaccacccccccgagcgctgctacgacttcaagatgtgcaaccgtttcaccgtggctctgcgttgccccgacggcgaagtgtgctactcccccgaaaagaccgctgagatccgtggtatcgtgaccaccatgacccactccctgacccgtcaggtggtgcacaacaagctgacctcttgcaactacaaccccctgtacctcgaggctgacggtcgtatccgttgcggcaaggtcaacgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccttggtccaccctgaccgctaaccagaacccctccccaccctggtccaagctgacctactccaagccccacgacgctgctaccttctactgccccttcctgtacccatccccccctcgttccccactgcagttctccggtttccagcgtgtgctgaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctcgagtcctgggctcacgtgttccgtgactactccgtgtccttccaagtgcgcctgaccttcaccgaggctaacaaccagacctacaccttctgcacccaccccaacctgatcgtgtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtctgtgccgtgtgtggctgtccgtgtgcctgtgcgctgtggtgctgggccagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccctcgagttctccgtgcgtctgttcgctaactaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 20 - DNA, 11811 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggtttgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgactccggcgccctggaagttctgttccaggggcccggaagcggcggaggggggtctggggggggaggcagccaccaccatcaccaccaccatcactaagaattcaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggggcctctaaacgggtcttgaggggttttttgctgaaaggaggaactatatccggatctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagctgacattcatccggggtcagcaccgtttctgcggactggctttctacgtgttccgcttcctttagcagcccttgcgccctgagtgcttgcggcagcgtgaagctaattctgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgtcccccaagaacctgacccccttcctgaccgctctgtggctgctgctgggccactcccgtgtgcctcgtgtgcgtgctgaggaatgctgcgagttcatcaacgtgaaccacccccccgagcgctgctacgacttcaagatgtgcaaccgtttcaccgtggctctgcgttgccccgacggcgaagtgtgctactcccccgaaaagaccgctgagatccgtggtatcgtgaccaccatgacccactccctgacccgtcaggtggtgcacaacaagctgacctcttgcaactacaaccccctgtacctcgaggctgacggtcgtatccgttgcggcaaggtcaacgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccttggtccaccctgaccgctaaccagaacccctccccaccctggtccaagctgacctactccaagccccacgacgctgctaccttctactgccccttcctgtacccatccccccctcgttccccactgcagttctccggtttccagcgtgtgctgaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctcgagtcctgggctcacgtgttccgtgactactccgtgtccttccaagtgcgcctgaccttcaccgaggctaacaaccagacctacaccttctgcacccaccccaacctgatcgtgtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtctgtgccgtgtgtggctgtccgtgtgcctgtgcgctgtggtgctgggccagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccctcgagttctccgtgcgtctgttcgctaactaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 21 - DNA, 11841 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggtttgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgactccggcgccctggaagttctgttccaggggcccccggtccgagagaacctgtacttccaaggcggaagcggcggaggggggtctggggggggaggcagccaccaccatcaccaccaccatcactaagaattcaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggggcctctaaacgggtcttgaggggttttttgctgaaaggaggaactatatccggatctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagctgacattcatccggggtcagcaccgtttctgcggactggctttctacgtgttccgcttcctttagcagcccttgcgccctgagtgcttgcggcagcgtgaagctaattctgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgtcccccaagaacctgacccccttcctgaccgctctgtggctgctgctgggccactcccgtgtgcctcgtgtgcgtgctgaggaatgctgcgagttcatcaacgtgaaccacccccccgagcgctgctacgacttcaagatgtgcaaccgtttcaccgtggctctgcgttgccccgacggcgaagtgtgctactcccccgaaaagaccgctgagatccgtggtatcgtgaccaccatgacccactccctgacccgtcaggtggtgcacaacaagctgacctcttgcaactacaaccccctgtacctcgaggctgacggtcgtatccgttgcggcaaggtcaacgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccttggtccaccctgaccgctaaccagaacccctccccaccctggtccaagctgacctactccaagccccacgacgctgctaccttctactgccccttcctgtacccatccccccctcgttccccactgcagttctccggtttccagcgtgtgctgaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctcgagtcctgggctcacgtgttccgtgactactccgtgtccttccaagtgcgcctgaccttcaccgaggctaacaaccagacctacaccttctgcacccaccccaacctgatcgtgtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtctgtgccgtgtgtggctgtccgtgtgcctgtgcgctgtggtgctgggccagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccctcgagttctccgtgcgtctgttcgctaactaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 22 - protein, 8 aa, artificial
  synthetic sequence
SIINFEKL

SEQ ID NO: 23 - protein, 29 aa, artificial
  synthetic sequence
AKFVAAWTLKAAAVAKXVAAWTLKAAAXC

SEQ ID NO: 24 - protein, 16 aa, artificial
  synthetic sequence
TAKFVAAWTLKAAAVG

SEQ ID NO: 25 - protein, 5 aa, artificial
  synthetic sequence
PADRE

SEQ ID NO: 26 - protein, 16 aa, artificial
  epitope derived from gB (UL55)
MQLMPDDYSNTHSTRY

SEQ ID NO: 27 - protein, 16 aa, artificial
  epitope derived from gB (UL55)
PDDYSNTHSTRYVTVK

SEQ ID NO: 28 - protein, 11 aa, artificial
  epitope derived from gB (UL55)
DYSNTHSTRYV

SEQ ID NO: 29 - protein, 16 aa, artificial
  epitope derived from gB (UL55)
TSAAHSRSGSVSQRVT

SEQ ID NO: 30 - protein, 16 aa, artificial
  epitope derived from gB (UL55)
HSRSGSVSQRVTSSQT

SEQ ID NO: 31 - protein, 16 aa, artificial
  epitope derived from gB (UL55)
GNVSVFETTGGLVVFW

SEQ ID NO: 32 - protein, 16 aa, artificial
  epitope derived from gB (UL55)
VFETTGGLVVFWQGIK

SEQ ID NO: 33 - protein, 15 aa, artificial
  epitope derived from gB (UL55)
FETTGGLVVFWQGIK

SEQ ID NO: 34 - protein, 13 aa, artificial
  epitope derived from gB (UL55)
YEYVDYLFKRMID

SEQ ID NO: 35 - protein, 20 aa, artificial
  epitope derived from gB (UL55)
NSAYEYVDYLFKRMIDLSSI

SEQ ID NO: 36 - protein, 20 aa, artificial
  epitope derived from gB (UL55)
MQLIPDDYSNTHSTRYVTVK

SEQ ID NO: 37 - protein, 15 aa, artificial
  epitope derived from IE-1
VKQIKVRVDMVRHRI

SEQ ID NO: 38 - protein, 15 aa, artificial
  epitope derived from IE-1
VRVDMVRHRIKEHML

SEQ ID NO: 39 - protein, 9 aa, artificial
  epitope derived from IE-1
TTFLQTMLR

SEQ ID NO: 40 - protein, 9 aa, artificial
  epitope derived from IE-1
KEVNSQLSL

SEQ ID NO: 41 - protein, 8 aa, artificial
  epitope derived from IE-1
QIKVRVDM

SEQ ID NO: 42 - protein, 9 aa, artificial
  epitope derived from IE-1
QIKVRVDMV

SEQ ID NO: 43 - protein, 9 aa, artificial
  epitope derived from IE-1
RIKEHMLKK

SEQ ID NO: 44 - protein, 9 aa, artificial
  epitope derived from IE-1
KLGGALQAK

SEQ ID NO: 45 - protein, 9 aa, artificial
  epitope derived from IE-1
ELRRKMMYM

SEQ ID NO: 46 - protein, 11 aa, artificial
  epitope derived from IE-1
FPKTTNGCSQA

SEQ ID NO: 47 - protein, 9 aa, artificial
  epitope derived from IE-1
AYAQKIFKI

SEQ ID NO: 48 - protein, 10 aa, artificial
  epitope derived from IE-1
TSDACMMTMY

SEQ ID NO: 49 - protein, 9 aa, artificial
  epitope derived from IE-1
CRVLCCYVL

SEQ ID NO: 50 - protein, 9 aa, artificial
  epitope derived from IE-1
VLEETSVML

SEQ ID NO: 51 - protein, 10 aa, artificial
  epitope derived from IE-1
YILGADPLRV

SEQ ID NO: 52 - protein, 9 aa, artificial
  epitope derived from IE-1
EEAIVAYTL

SEQ ID NO: 53 - protein, 15 aa, artificial
  epitope derived from IE-2
TRRGRVKIDEVSRMF

SEQ ID NO: 54 - protein, 15 aa, artificial
  epitope derived from pp65
DQYVKVYLESFCEDV

SEQ ID NO: 55 - protein, 15 aa, artificial
  epitope derived from pp65
GKISHIMLDVAFTSH

SEQ ID NO: 56 - protein, 15 aa, artificial
  epitope derived from pp65
EHPTFTSQYRIQGKL

SEQ ID NO: 57 - protein, 15 aa, artificial
  epitope derived from pp65
AGILARNLVPMVATV

SEQ ID NO: 58 - protein, 15 aa, artificial
  epitope derived from pp65
GQNLKYQEFFWDAND

SEQ ID NO: 59 - protein, 15 aa, artificial
  epitope derived from pp65
KYQEFFWDANDIYRI

SEQ ID NO: 60 - protein, 9 aa, artificial
  epitope derived from pp65
NLVPMVATV

SEQ ID NO: 61 - protein, 12 aa, artificial
  epitope derived from gH
HELLVLVKKAQL

SEQ ID NO: 62 - protein, 11 aa, artificial
  epitope derived from gH
HELLVLVKKAQ

SEQ ID NO: 63 - protein, 561 aa, Cytomegalovirus
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 64 - protein, 907 aa, Cytomegalovirus
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSINVFDLEEIMREFNSYKQRVKYVEDKVVDPLPPYLKGLDDLMSGLGAAGKAVGVAIGAVGGAVASVVEGVATFLKNPFGAFTIILVAIAVVIIIYLIYTRQRRLCMQPLQNLFPYLVSADGTTVTSGNTKDTSLQAPPSYEESVYNSGRKGPGPPSSDASTAAPPYTNEQAYQMLLALVRLDAEQRAQQNGTDSLDGQTGTQDKGQKPNLLDRLRHRKNGYRHLKDSDEEENV

SEQ ID NO: 65 - protein, 491 aa, Cytomegalovirus
MESSAKRKMDPDNPDEGPSSKVPRPETPVTKATTFLQTMLRKEVNSQLSLGDPLFPELAEESLKTFERVTEDCNENPEKDVLAELVKQIKVRVDMVRHRIKEHMLKKYTQTEEKFTGAFNMMGGCLQNALDILDKVHEPFEEMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMAYAQKIFKILDEERDKVLTHIDHIFMDILTTCVETMCNEYKVTSDACMMTMYGGISLLSEFCRVLCCYVLEETSVMLAKRPLITKPEVISVMKRRIEEICMKVFAQYILGADPLRVCSPSVDDLRAIAEESDEEEAIVAYTLATAGASSSDSLVSPPESPVPATIPLSSVIVAENSDQEESEQSDEEEEEGAQEEREDTVSVKSEPVSEIEEVAPEEEEDGAEEPTASGGKSTHPMVTRSKADQ

SEQ ID NO: 66 - protein, 579 aa, Cytomegalovirus
MESSAKRKMDPDNPDEGPSSKVPRPETPVTKATTFLQTMLRKEVNSQLSLGDPLFPELAEESLKTFERVTEDCNENPEKDVLAELGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMHQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 67 - DNA, 9449 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgaactccaaggacctgacccccttcctgaccaccctgtggctgctgctggaccactcccgtgtgcctcgtgtgcgtgctgaggaatgctgcgaagtgatcaacgtgaaccacccccccgagcgctgctacgacttcaagatgtgcaacctgttcaccgtggctctgcgttgccccgacggcgaagtgtgctactcccccgaaaagaccgctgagatccgtggtatcgtgaccaccatgacccactccctgacccgtcaagtgatccacaacaagctgaccagctgcaactacaaccccctgtacctcgaggctgacggtcgtatccgttgcggcaaggtgtccgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccctggttcaccctgaccgctaaccagaacccctccccaccctggtccaagctgacctaccccaagccccacgacgctgctaccttctactgccccttcctgtacccatccccacctcgttccccatcccagttctccggtttccagcgtgtgtccaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctggaatcctgggctcacgtgttccgtgactactccgtgtccttccaagtgcgcctgaccttcaccgaggctaacaaccagacctacaccttctgcacccaccccaacctgatcgtgtaagtcgacaagcttaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtctgtgccgtgtgtggctgtccgtgtgcctgtgcgctgtggtgctgggacagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccttggagttctccgtgcgtctgttcgccaactaataaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccccgggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgactccggcgccctggaagttctgttccaggggcccggaagcggcggaggggggtctggggggggaggcagccaccaccatcaccaccaccatcactaagaattcaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 68 - DNA, 9449 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgtcccccaagaacctgacccccttcctgaccgctctgtggctgctgctgggccactcccgtgtgcctcgtgtgcgtgctgaggaatgctgcgagttcatcaacgtgaaccacccccccgagcgctgctacgacttcaagatgtgcaaccgtttcaccgtggctctgcgttgccccgacggcgaagtgtgctactcccccgaaaagaccgctgagatccgtggtatcgtgaccaccatgacccactccctgacccgtcaggtggtgcacaacaagctgacctcttgcaactacaaccccctgtacctcgaggctgacggtcgtatccgttgcggcaaggtcaacgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccctggttcaccctgaccgctaaccagaacccctccccaccctggtccaagctgacctaccccaagccccacgacgctgctaccttctactgccccttcctgtacccatccccacctcgttccccatcccagttctccggtttccagcgtgtgtccaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctggaatcctgggctcacgtgttccgtgactactccgtgtccttccaagtgcgcctgaccttcaccgaggctaacaaccagacctacaccttctgcacccaccccaacctgatcgtgtaagtcgacaagcttaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtctgtgccgtgtgtggctgtccgtgtgcctgtgcgctgtggtgctgggacagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccttggagttctccgtgcgtctgttcgccaactaataaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccccgggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgactccggcgccctggaagttctgttccaggggcccggaagcggcggaggggggtctggggggggaggcagccaccaccatcaccaccaccatcactaagaattcaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 69 - DNA, 8986 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgaactccaaggacctgacccccttcctgaccaccctgtggctgctgctggaccactcccgtgtgcctcgtgtgcgtgctgaggaatgctgcgaagtgatcaacgtgaaccacccccccgagcgctgctacgacttcaagatgtgcaacctgttcaccgtggctctgcgttgccccgacggcgaagtgtgctactcccccgaaaagaccgctgagatccgtggtatcgtgaccaccatgacccactccctgacccgtcaagtgatccacaacaagctgaccagctgcaactacaaccccctgtacctcgaggctgacggtcgtatccgttgcggcaaggtgtccgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccctggttcaccctgaccgctaaccagaacccctccccaccctggtccaagctgacctaccccaagccccacgacgctgctaccttctactgccccttcctgtacccatccccacctcgttccccatcccagttctccggtttccagcgtgtgtccaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctggaatcctgggctcacgtgttccgtgactactccgtgtccttccaagtgcgcctgaccttcaccgaggctaacaaccagacctacaccttctgcacccaccccaacctgatcgtgtaagtcgacaagcttaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtctgtgccgtgtgtggctgtccgtgtgcctgtgcgctgtggtgctgggacagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccttggagttctccgtgcgtctgttcgccaaccagctgttgaattttgaccttcttaagcttgcgggagacgtcgagtccaaccctgggcccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgactccggcgccctggaagttctgttccaggggcccggaagcggcggaggggggtctggggggggaggcagccaccaccatcaccaccaccatcactaagaattcaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 70 - DNA, 8986 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgtcccccaagaacctgacccccttcctgaccgctctgtggctgctgctgggccactcccgtgtgcctcgtgtgcgtgctgaggaatgctgcgagttcatcaacgtgaaccacccccccgagcgctgctacgacttcaagatgtgcaaccgtttcaccgtggctctgcgttgccccgacggcgaagtgtgctactcccccgaaaagaccgctgagatccgtggtatcgtgaccaccatgacccactccctgacccgtcaggtggtgcacaacaagctgacctcttgcaactacaaccccctgtacctcgaggctgacggtcgtatccgttgcggcaaggtcaacgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgtggctagctttgtttaactttaagaaggagatacatctagaccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgctgcgtctgctgctgcgtcaccacttccactgcctgctgctgtgcgctgtgtgggctaccccctgcctggcttccccctggttcaccctgaccgctaaccagaacccctccccaccctggtccaagctgacctaccccaagccccacgacgctgctaccttctactgccccttcctgtacccatccccacctcgttccccatcccagttctccggtttccagcgtgtgtccaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctggaatcctgggctcacgtgttccgtgactactccgtgtccttccaagtgcgcctgaccttcaccgaggctaacaaccagacctacaccttctgcacccaccccaacctgatcgtgtaagtcgacaagcttaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgggtgcctaatgagtgagctaactcacattaattgcccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgcgtctgtgccgtgtgtggctgtccgtgtgcctgtgcgctgtggtgctgggacagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccttggagttctccgtgcgtctgttcgccaaccagctgttgaattttgaccttcttaagcttgcgggagacgtcgagtccaaccctgggcccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgactccggcgccctggaagttctgttccaggggcccggaagcggcggaggggggtctggggggggaggcagccaccaccatcaccaccaccatcactaagaattcaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 71 - DNA, 8038 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgaactccaaggacctgacccccttcctgaccaccctgtggctgctgctggaccactcccgtgtgcctcgtgtgcgtgctgaggaatgctgcgaagtgatcaacgtgaaccacccccccgagcgctgctacgacttcaagatgtgcaacctgttcaccgtggctctgcgttgccccgacggcgaagtgtgctactcccccgaaaagaccgctgagatccgtggtatcgtgaccaccatgacccactccctgacccgtcaagtgatccacaacaagctgaccagctgcaactacaaccccctgtacctcgaggctgacggtcgtatccgttgcggcaaggtgtccgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagcagctgttgaattttgaccttcttaagcttgcgggagacgtcgagtccaaccctgggcccatgctgcgtctgctgctgcgtcaccacttccactgcctgctgctgtgcgctgtgtgggctaccccctgcctggcttccccctggttcaccctgaccgctaaccagaacccctccccaccctggtccaagctgacctaccccaagccccacgacgctgctaccttctactgccccttcctgtacccatccccacctcgttccccatcccagttctccggtttccagcgtgtgtccaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctggaatcctgggctcacgtgttccgtgactactccgtgtccttccaagtgcgcctgaccttcaccgaggctaacaaccagacctacaccttctgcacccaccccaacctgatcgtgcagctgttgaattttgaccttcttaagcttgcgggagacgtcgagtccaaccctgggcccatgcgtctgtgccgtgtgtggctgtccgtgtgcctgtgcgctgtggtgctgggacagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccttggagttctccgtgcgtctgttcgccaaccagctgttgaattttgaccttcttaagcttgcgggagacgtcgagtccaaccctgggcccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgactccggcgccctggaagttctgttccaggggcccggaagcggcggaggggggtctggggggggaggcagccaccaccatcaccaccaccatcactaagaattcaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 72 - DNA, 8038 bp, artificial
  synthetic sequence for expression of pentameric CMV complex
ttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgcccttgcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacagctttgtttgtactatcaacaggttgaactgctgatcaacagatcctctacgcggccgcggtaccataacttcgtatagcatacattatacgaagttatctggtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttagcgagcgtccacagcctggggaccgtaacgggagtgagcgggcaggttcacacgggtctgcttcagctcagggggcaggccagcgtagtacttgtccaggtgacgcagcaggggggggtccagctggtgacgcaggcagaattccttcacagcgttgtacaggccgtagaacagggtgataccctcgggagcagcagcggtggacacgggcagacgcacagcacggttggtacgggtagcctcgttacggatagccaccaccacgttgaacagggaggggggcaccagctcgaaacccagcacgtgttcggtgaagatggaacgaccgtaggacagacgagtcaggtcgtaaccacggcacaggtcgtccacgcaagtgtacacagcgggggaaccgtcgccgcactcggagtaaccgcgcatcacggtcatccaacggggagcggtgtcggaggacagcagggtcagcagagcacgcagctggtcggggttgttgtacagcagggccagggtgtccaggaaagcgtcgtccagcagcacggagttagcagcctcgggggtcacgggacggtaacggatcagctgggacaggggaccgtccctacgggtcacgttgaccaggggacgcagccaggactcgtacttgtcgccctggaacacctctcccagcaggcaacgacgggtcagctcggggcactcagcgggcaccttctcagccacggtgggagccacggacacggtagcggaggacacgatgggcagcagcaggcagcaccacagcagagccacgggaccgggggagaaggagaaaccgcagtcgggacgacggcacatggtggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccgccaccatgtcccccaagaacctgacccccttcctgaccgctctgtggctgctgctgggccactcccgtgtgcctcgtgtgcgtgctgaggaatgctgcgagttcatcaacgtgaaccacccccccgagcgctgctacgacttcaagatgtgcaaccgtttcaccgtggctctgcgttgccccgacggcgaagtgtgctactcccccgaaaagaccgctgagatccgtggtatcgtgaccaccatgacccactccctgacccgtcaggtggtgcacaacaagctgacctcttgcaactacaaccccctgtacctcgaggctgacggtcgtatccgttgcggcaaggtcaacgacaaggctcagtacctgctgggtgctgctggttccgtgccctaccgttggatcaacctcgagtacgacaagatcacccgtatcgtgggcctggaccagtacctcgagtccgtgaagaagcacaagcgtctggacgtgtgccgtgctaagatgggttacatgctgcagcagctgttgaattttgaccttcttaagcttgcgggagacgtcgagtccaaccctgggcccatgctgcgtctgctgctgcgtcaccacttccactgcctgctgctgtgcgctgtgtgggctaccccctgcctggcttccccctggttcaccctgaccgctaaccagaacccctccccaccctggtccaagctgacctaccccaagccccacgacgctgctaccttctactgccccttcctgtacccatccccacctcgttccccatcccagttctccggtttccagcgtgtgtccaccggtcccgagtgccgtaacgagactctgtacctgctgtacaaccgcgagggccagaccctggtcgagaggtcctccacctgggtcaagaaggtcatctggtacttgtccggtcgtaaccagaccatcctgcagcgtatgccccgtaccgcttccaagccctccgacggcaacgtgcagatctccgtcgaggacgctaagatcttcggtgctcacatggtgcccaagcagaccaagctgctgcgtttcgtggtcaacgacggcacccgttaccagatgtgcgtgatgaagctggaatcctgggctcacgtgttccgtgactactccgtgtccttccaagtgcgcctgaccttcaccgaggctaacaaccagacctacaccttctgcacccaccccaacctgatcgtgcagctgttgaattttgaccttcttaagcttgcgggagacgtcgagtccaaccctgggcccatgcgtctgtgccgtgtgtggctgtccgtgtgcctgtgcgctgtggtgctgggacagtgccagcgtgaaaccgctgagaagaacgactactaccgtgtgccccactactgggacgcttgctcccgtgctctgcccgaccagacccgttacaagtacgtcgagcagctggtggacctgaccctgaactaccactacgacgcttcccacggcctggacaacttcgacgtgctgaagcgtatcaacgtgaccgaggtgtccctgctgatctccgacttccgtcgtcagaaccgtcgtggtggcaccaacaagcgtaccaccttcaacgctgctggttccctggctccccacgctcgttccttggagttctccgtgcgtctgttcgccaaccagctgttgaattttgaccttcttaagcttgcgggagacgtcgagtccaaccctgggcccatgcgtcccggcctgccctcctacctgatcgtgctggctgtgtgcctgctgtcccacctcctgtcctcccgttacggtgctgaggctatctccgagcccctggacaaggctttccacctcctgctgaacacctacggtcgtcctatccgtttcctgcgcgagaacaccacccagtgcacctacaactcctccctgcgcaactccaccgtcgtccgcgagaacgctatctccttcaacttcttccagtcctacaaccagtactacgtgttccacatgccccgttgcctgttcgctggtcccctggctgagcagttcctgaaccaggtggacctgaccgagactctcgagcgttaccagcagcgtctgaacacttacgctctggtgtccaaggacctggcttcctaccgttccttcagccagcagctgaaggctcaggactccctgggcgagcagcccaccactgtgcctccccctatcgacctgtccatcccccacgtgtggatgcccccccagaccactcctcacggctggaccgagtcccacaccacctccggactgcaccgtccccacttcaaccagacctgcatcctgttcgacggtcacgacctgctgttctccaccgtgaccccctgcctgcaccagggcttctacctcatcgacgagctgcgttacgtgaagatcaccctgaccgaggatttcttcgtggtcaccgtgtccatcgacgacgacacccccatgctgctgatcttcggtcacctcccccgtgtgctgttcaaggctccctaccagcgtgacaacttcatcctgcgtcagaccgagaagcacgagctgctggtgctggtcaagaaggaccagctgaaccgtcactcttacctgaaggaccccgacttcctggacgctgctctggacttcaactacctggacctgtccgctctgctgaggaactccttccaccgttacgctgtggacgtgctgaagtccggtcgttgccagatgctggaccgtcgtaccgtcgagatggctttcgcttacgccctggctctgttcgctgctgctcgtcaagaagaggctggcgctcaggtgtccgtgccccgtgctctggaccgtcaggctgctctgctgcagatccaagagttcatgatcacttgcctgtcccagaccccccctcgtaccaccctgctgctgtaccccaccgctgtggacctggctaagcgtgctctgtggacccccaaccagatcaccgacatcacctccctcgtgcgtctggtgtacatcctgtccaagcagaaccagcagcacctgatcccccagtgggctctgcgtcagatcgctgacttcgctctgaagctgcacaagacccacctggcttcattcctcagcgctttcgctcgccaagagctgtacctgatgggttccctggtgcactccatgctggtgcacaccaccgagcgtcgcgagatcttcatcgtggaaaccggcctgtgctccctggccgagctgtcccacttcacccagctgctggctcacccccaccacgagtacctgtccgacctgtacaccccctgctcctcttccggtcgtcgtgaccactccctggaacgtctgacccgtctgttccccgacgctaccgtgcctaccaccgtgcctgctgctctgtccatcctgtctaccatgcagccctccaccctggaaaccttccccgacctgttctgcctgcctctgggcgagtccttctccgctctgaccgtgtccgagcacgtgtcctacgtggtcaccaaccagtacctgatcaagggcatctcctaccccgtgtccaccaccgtcgtgggccagtccctgatcatcacccagaccgactcccagaccaagtgcgagctgacccgtaacatgcacaccactcactccatcaccgctgctctgaacatctccctcgagaactgcgctttctgccagtccgctctgttggagtacgacgacactcagggtgtcatcaacatcatgtacatgcacgactccgacgacgtgctgttcgctctggacccctacaacgaggtggtggtgtcctccccccgtacccactacctgatgctgctgaagaacggcaccgtgctggaagtgaccgacgtggtggtggacgctaccgactccggcgccctggaagttctgttccaggggcccggaagcggcggaggggggtctggggggggaggcagccaccaccatcaccaccaccatcactaagaattcaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca